(19) United States
(12) Patent Application Publication
(10) Pub. No.: US 2024/0358672 A1
(43) Pub. Date: Oct. 31, 2024

(54) APPLICATION OF GENISTEIN AND ITS PHOSPHATE ESTER DERIVATIVE AND PHARMACEUTICAL COMPOSITION COMPRISING THE SAME

(71) Applicant: National Taiwan University, Taipei (TW)

(72) Inventors: NAN-WEI SU, Taipei (TW); CHEN HSU, Taipei (TW); Tzu-Tang WEI, Taipei (TW); Dai-Jung CHUNG, Taipei (TW)

(21) Appl. No.: 18/309,560

(22) Filed: Apr. 28, 2023

(51) Int. Cl. A61K 31/352; A61P 3/00

(52) U.S. Cl. CPC A61K 31/352; A61P 3/00

(57) ABSTRACT

The present invention provides an application of genistein and its phosphate ester derivative for treating or preventing diseases mediated by the cannabinoid receptor type 1 (CB1 receptor) in an individual. The present invention also provides a pharmaceutical composition for treating or preventing CB1 receptor-mediated diseases, comprising an effective amount of genistein and its phosphate ester derivative.

### BACKGROUND OF THE INVENTION

### 1. Technical Field

The present invention relates to the field of pharmaceutical technology and pertains to a pharmaceutical composition comprising genistein and its phosphate ester derivatives, as well as their uses, particularly for the treatment or prevention of CB1 receptor-mediated diseases.

### 2. Description of Related Art

Marijuana is one of the most widely used illicit drugs worldwide. The growing legalization of marijuana is expected to increase the use of marijuana, highlighting the need to learn about its adverse effects, particularly those affecting the cardiovascular systems.

Retrospective studies indicate that marijuana increases the risk of cardiovascular disease (CVD), including myocardial infarction (MI), angina, and arrhythmias. Marijuana exposure is known to induce endothelial dysfunction, atherosclerosis, cardiomyopathy, and metabolic dysfunction in animal models. Nevertheless, the US Food and Drug Administration (FDA) has approved two synthetic cannabinoids for treating chemotherapy-induced nausea and vomiting and HIV-associated anorexia: dronabinol and nabilone (Whiting et al., 2015). However, synthetic cannabinoids are also associated with adverse cardiovascular effects (Pacher et al., 2018).

The main active ingredient contained in marijuana is Δ⁹-tetrahydrocannabinol (Δ⁹-THC), which interacts with specific receptors in the human body, such as: cannabinoid receptor 1 (CB1 receptor) and cannabinoid receptor 2 (CB2 receptor), both of which belong to the G-protein-coupled receptor (GPCR) superfamily. In addition to exogenous Δ⁹-THC, the endocannabinoid system (ECS) that exists naturally in the human body also has a similar effect. The endocannabinoid anandamide is made on-demand, unlike classical neurotransmitters, and causes vasodilation, bradycardia, and hypotension (Movahed et al., 2005). By binding to the cannabinoid receptors, endocannabinoids can play a role in regulating physiological functions such as metabolism, memory, emotion, pain and immunity in the body.

In addition to being abundantly expressed in the brain, CB1 receptor is also expressed in other peripheral tissues such as the heart, vasculature, and smooth muscle. Previous studies found that CB1 receptor activation is proatherogenic by promoting inflammation and oxidative stress that cause endothelial dysfunction and atherosclerosis. Therefore, the development of antagonists of CB1 receptor can be used for anti-atherosclerosis (Pacher et al., 2018; Ibsen et al., 2017).

CB1 receptor signaling is also involved in various pathophysiological processes, including obesity, smoking cessation, diabetes, liver cirrhosis, and cancer (Sugamura et al., 2009). In 2006, rimonabant became the first CB1 antagonist approved for treating obesity (Després et al., 2005), which has a good effect on weight loss and improvement of metabolic symptoms. However, because rimonabant can penetrate the blood-brain barrier (BBB), its negative effect on the brain and nervous system would cause some patients have serious psychiatric side effects such as anxiety, depression, and even suicidal tendencies, which made the drug unsuccessful in marketing in the United States, and was even withdrawn in Europe in 2008 (Onakpoya et al., 2016; Moreira et al., 2009). Therefore, there is an urgent need to develop CB1 antagonists that lack psychiatric side effects would be clinically significant for treating obesity, diabetes, metabolic syndrome, and CVD.

### BRIEF SUMMARY OF THE INVENTION

This part of the specification aims to provide a brief summary of the invention so as to enable a basic understanding of the invention. The brief summary of the invention is neither a complete description of the invention nor intended to point out the important or key elements of certain embodiments of the invention or define the scope of the invention.

Based on the prior art, it is known that cannabinoids may increase the risk of cardiovascular diseases. Specifically, this may be attributed to the binding of cannabinoids to CB1 receptors, causing vascular inflammation, oxidative stress, and atherosclerosis. In view of this, the inventors of the present invention believe that CB1 antagonists that do not induce negative effects on brain neurons are of crucial importance.

In view of the demand for CB1 antagonists, the present invention uses genistein in soybean isoflavones as a lead drug to alleviate atherosclerosis induced by Δ⁹-THC, and develops a new generation of CB1 receptor antagonists, which can be used as a drug for CB1 receptor-related diseases.

An aspect of the present invention provides a use of a genistein and its phosphate ester derivative for the preparation of a medicament for treatment or prevention of diseases mediated by cannabinoid receptor type 1 (CB1 receptor) in an individual.

In some embodiments, the treatment or prevention of CB1 receptor-mediated diseases in the individual involves alleviating oxidative stress and endothelial dysfunction induced by Δ⁹-tetrahydrocannabinol, dronabinol, or nabilone.

In some embodiments, the oxidative stress involves a decrease in the expression levels of antioxidant genes and an increase in the expression levels of reactive oxygen species (ROS)-related genes: the antioxidant genes include superoxide dismutase 1 (SOD1) gene, superoxide dismutase 2 (SOD2) gene, catalase (CAT) gene, or glutathione peroxidase 1 (GPX1) gene, and the ROS-related genes involve the expression of NADPH oxidase 1 (NOX1) or inducible nitric oxide synthase (NOS2).

In some embodiments, the medicament alleviates inflammation induced by Δ⁹-tetrahydrocannabinol, dronabinol, or nabilone.

In some embodiments, the medicament reduces mRNA expression levels of pro-inflammatory cytokines and chemokines induced by Δ⁹-tetrahydrocannabinol, dronabinol, or nabilone.

In some embodiments, the CB1 receptor-mediated diseases are diseases or conditions associated with the coupling of CB1 receptor with Δ⁹-tetrahydrocannabinol, dronabinol, or nabilone.

In some embodiments, the CB1 receptor-mediated diseases include cardiovascular diseases, mental disorders, anxiety disorders, depression, epilepsy, neurodegenerative diseases, cognitive impairment, brain trauma, glaucoma, Parkinson's disease, Alzheimer's disease, Huntington's chorea, tremors, senile dementia, Tourette syndrome, cancer, hypotonia, septic shock, hypotension, multiple sclerosis, vomiting, asthma, eating disorders, obesity, diabetes, diseases associated with demyelination, neuroinflammation, viral encephalitis, liver cirrhosis, or gastrointestinal diseases.

In some embodiments, the cardiovascular diseases include atherosclerosis, coronary heart disease, peripheral vascular disease, myocardial infarction, and carotid artery stenosis.

Another aspect of the present invention provides a pharmaceutical composition for treatment or prevention of CB1 receptor-mediated diseases, comprising an effective amount of genistein and its phosphate ester derivative.

In some embodiments, the pharmaceutical composition further comprises at least one other therapeutic agent.

In some embodiments, the at least one other therapeutic agent is anti-obesity agents, appetite suppressants, anti-diabetic agents, anti-hyperlipidemic agents, lipid-lowering agents, blood cholesterol-lowering agents, fat-modulating agents, cholesterol-lowering agents, fat-reducing agents, high-density lipoprotein-raising agents, anti-hypertensive agents, preparations for treating sleep disorders, preparations for treating substance abuse or addiction disorders, anti-anxiety agents, antidepressants, antipsychotic agents, cognitive enhancers, preparations for treating cognitive disorders, preparations for treating Alzheimer's disease, preparations for treating Parkinson's disease, anti-inflammatory agents, preparations for treating neurodegenerative disorders, preparations for treating arteriosclerosis, preparations for treating respiratory conditions, preparations for treating intestinal diseases, preparations for treating liver cirrhosis, or antineoplastic agents.

In some embodiments, the pharmaceutical composition is used for the treatment of chronic dependence, alcohol dependence, or drug abuse.

In some embodiments, the pharmaceutical composition is used for the treatment of cannabis abuse.

In some embodiments, the pharmaceutical composition enhances the analgesic activity of analgesic drugs or anesthetic drugs.

In some embodiments, the phosphate ester derivative of genistein is genistein 7-O-phosphate (G7P) or genistein 4′-O-phosphate (G4′P).

In some embodiments, the phosphate ester derivative of genistein is obtained by microbial fermentation of genistein or by contacting or culturing genistein with a polyphenolic flavonoid phosphate synthase.

In some embodiments, the microorganism comprises a nucleic acid sequence encoding a polypeptide having a homologous protein sequence with a similarity of at least 70% to the polyphenolic flavonoid phosphate synthase (SEQ ID NO: 1), said polypeptide comprising a conserved region, said conserved region being based on SEQ ID NO: 1 and sequentially comprising: an ATP binding region, including active catalytic sites of lysine 27 (Lys27), arginine 102 (Arg102), and glutamic acid 282 (Glu282): a substrate binding region, including a conserved motif DDHHFYIDAMLDAKAR (SEQ ID NO: 2), and active catalytic sites of aspartic acid 627 (Asp627), histidine 629 (His629), and histidine 630 (His630); and a phosphorylated histidine catalytic region, including an active catalytic site of histidine 795 (His795).

Genistein and its phosphate ester derivatives of the present invention can bind to CB1 receptors and exert preventive and therapeutic effects on CB1 receptor-mediated diseases without penetrating the blood-brain barrier, thereby improves the shortcomings of previous drugs and represents a new generation of superior CB1 receptor antagonists. Additionally, the present invention utilizes the phosphate ester derivatives of genistein to enhance bioavailability and water solubility, further enhancing the efficacy of genistein.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

In order to make the above-mentioned present invention and other objects, features, advantages and embodiments more obvious and understandable, its drawings are described as follows.

FIG. 1A to 1D illustrate the screening results of CB1 receptor antagonists according to Example 1. FIG. 1A: Query structure of 4 selective CB1 antagonists (rimonabant, otenabant, AM251, and DBPR-211) was used in the ligand-based high-throughput virtual screening. FIG. 1B: Docking of the selective CB1 antagonist AM6538 and genistein into the CB1 receptor. The best fit of AM6538 and genistein into CB1 was shown using Schrödinger molecule docking software. FIG. 1C: Genistein shared structural homology with selective CB1 antagonists in ligand-based virtual screening. FIG. 1D: Genistein is a neutral antagonist of the CB1 receptor. GTPase-Glo assay reveals rimonabant and taranabant to decrease GTP turnover compared with Apo and GTPase without ligand, indicating that they are inverse agonists. Genistein elicits the same GTP turnover as Apo, suggesting that genistein is a neutral antagonist. CP55490 causes increased GTP turnover consistent with its function as a CB1 agonist.

FIG. 2A to 2G illustrate the effects of Δ⁹-THC on the cytotoxicity, inflammation-related genes and oxidative stress protection-related genes of endothelial cells/artificially induced pluripotent stem cells-endothelial cells (hiPSC-EC) according to Example 2 and 3. FIG. 2A: The effects of Δ⁹-THC on cell viability of human coronary artery endothelial cells (HCAECs), human umbilical vein endothelial cells (HUVECs), normal human cardiac fibroblasts-ventricular (NHCF-V), and human embryonic stem cell-derived cardiomyocytes (hESC-CMs). Cells were treated with increasing concentrations of Δ⁹-THC for 48 hours, and cell viability was measured by the CellTiter-Glo luminescent cell viability assay. FIGS. 2B and 2C: The RNA expression of inflammation-related (B) and oxidative stress-related genes (C) in HUVECs. Cells were treated with 5 μM Δ⁹-THC for 48 hours, and gene expression was measured by qPCR and normalized to GAPDH. FIG. 2D: Cells were treated with various concentrations of D⁹-THC for 24 or 48 hours, and the CellTiter-Glo luminescent cell viability assay measured cell viability. The arrowhead indicates the concentration used in the embodiment. FIG. 2E: Oxidative stress-related gene expression in hiPSC-ECs after Δ⁹-THC treatment. Cells were treated with 5 μM Δ⁹-THC for 48 hours, and mRNA expression of various genes was measured by qPCR analysis and normalized to GAPDH. FIG. 2F: hiPSC-ECs were treated with 5 μM Δ⁹-THC or 1 μM doxorubicin (positive control) for 48 hours and oxidative stress was measured by CellROX oxidative stress assay. Images were obtained by fluorescence microscopy. The white arrowhead indicates cells producing reactive oxygen species (scale bar, 50 μm). FIG. 2G: Δ⁹-THC induced hydrogen peroxide (H₂O₂) production in hiPSC-ECs. hiPSC-ECs were treated with 0.5 or 5 μM Δ⁹-THC for 48 hours, and ROS-Glo™ H₂O₂ assay measured the level of hydrogen peroxide.

FIG. 3A to 3D illustrate the effect of Δ⁹-THC on the inflammatory response of hiPSC-EC according to Example 4. FIG. 3A: RNA expression of inflammation-related genes in hiPSC-ECs after Δ⁹-THC treatment for 48 hours, as determined by qPCR and normalized to GAPDH. FIG. 3B: Δ⁹-THC promoted the release of TNF-α from hiPSC-ECs. Cells were treated with 0.5 or 5 μM Δ⁹-THC for 48 hours, and TNF-α concentration in the cell culture medium was measured by ELISA. FIG. 3C: Monocyte adhesion assays of hiPSC-ECs treated with 5 μM Δ⁹-THC for 48 hours. U937 adherence was observed by a fluorescence microscope (left panel). The intensity of fluorescence-labeled-adherent U937 monocytes was measured (right panel). FIG. 3D: The effect of Δ⁹-THC on the inflammation-related gene expression in hiPSC-ECs. Cells were treated with 5 μM Δ⁹-THC for 48 hours. The medium was replaced with fresh cell culture medium, and total RNA was isolated from cells every other day. Expression of inflammation-related genes was quantified by qPCR analysis and normalized to GAPDH.

FIG. 4A to 4D illustrate the mechanism of oxidative stress induced by Δ⁹-THC through CB1 receptors according to Example 5. FIG. 4A: Genistein decreased Δ⁹-THC-induced p38 phosphorylation in hiPSC-ECs. Cells were treated with 5 μM Δ⁹-THC, 10 μM genistein, or their combination for 48 hours. Total cell lysates were subjected to western blot analysis with anti-p38 antibody and anti-p38 phospho-Thr180/Tyr182 (p38 pThr180/Tyr182) antibody. Densitometry of p38 phospho-Thr180/Tyr182 expression was normalized to pan p38 expression. FIG. 4B: The effects of Δ⁹-THC on p65 nuclear translocation in hiPSC-ECs were determined in co-localization studies. hiPSC-ECs were treated with 5 μM Δ⁹-THC for 48 hours and then assayed with anti-p65 antibody for localization of p65 (red fluorescence). The nuclei were counterstained with DAPI (blue fluorescence), and cells were visualized using immunofluorescence microscopy: merged images are shown. The white arrowhead indicates co-localization (purple) of p65 (red) and DAPI (blue) (scale bar, 50 μm). FIG. 4C: Δ⁹-THC caused p65 phosphorylation in hiPSC-ECs. Cells were treated with 5 μM Δ⁹-THC, 1 μM BAY11-7082, or their combination for 48 hours. Western blot analysis was performed on total cell lysates using indicated antibodies (left panel). Images were quantified by ImageJ software (right panel). FIG. 4D: Cells were treated with 5 μM Δ⁹-THC, 1 μM BAY11-7082, or their combination for 48 hours. The mRNA expression of inflammation-related genes in hiPSC-ECs was quantified by qPCR analysis and normalized to GAPDH.

FIG. 5A to 5D illustrate the effect of CB1 receptors on the action of Δ⁹-THC and genistein according to Example 6. FIG. 5A: The selective CB1 antagonists AM6545, AM251, and NESS-0327 blocked the expression of inflammation-related genes and oxidative stress protective-related genes. Gene expression of inflammation-related genes and oxidative stress protective-related genes in hiPSC-ECs were quantified by qPCR analysis. hiPSC-ECs were treated with 5 μM Δ⁹-THC and either 3 μM AM6545, 3 μM AM251, or 1 μM NESS-0327, or their combination of 5 μM Δ⁹-THC and CB1 antagonist for 48 hours, and the gene expression was normalized to GAPDH. FIG. 5B: The effect of siRNA-mediated knockdown of CB1 on Δ⁹-THC-induced inflammation and oxidative stress in hiPSC-ECs. The hiPSC-ECs were treated with 5 μM Δ⁹-THC for 48 hours. The mRNA expression of inflammation-related genes and oxidative stress protective-related genes was quantified by qPCR analysis and normalized to GAPDH. FIG. 5C: hiPSC-ECs were treated with 5 μM Δ⁹-THC for 48 hours. The mRNA expression of inflammation-related genes and oxidative stress protective-related genes in hiPSC-ECs treated with sgRNA versus control was quantified by qPCR analysis and normalized to GAPDH. FIG. 5D: Wild-type (WT) hiPSC-ECs and CRISPRi edited hiPSC-ECs were incubated with 0 or 10 ng/ml TNF-α either alone or in the presence of 10 μM genistein. The expression of ILIA decreased with cotreatment of TNF-α and genistein in WT hiPSC-ECs but increased significantly in CB1 CRISPRi hiPSC-ECs. The expression of IL6 increased with TNF-α for WT and CRISPRi hiPSC-ECs but was attenuated by cotreatment with genistein in the WT hiPSC-ECs. The expression of CXCR8 increased with TNF-α for WT and CRISPRi hiPSC-ECs but was attenuated in WT hiPSC-ECs. The expression of SOD1 increased in response to TNF-α and was not attenuated by genistein in either WT or CB1 CRISPRi hiPSC-ECs.

FIG. 6A to 6I illustrate the effects of genistein on the responses induced by Δ⁹-THC according to Example 7. FIG. 6A: Compounds that mitigate reactive oxygen species production caused by Δ⁹-THC in hiPSC-ECs were screened. The cells were treated with 5 μM Δ⁹-THC plus antioxidant reagent or vehicle control for 48 hours, and the level of hydrogen peroxide was measured by ROS-Glo™ H₂O₂ assay. FIG. 6B: hiPSC-ECs were treated with 5 μM Δ⁹-THC and various antioxidant reagents for 48 hours, and CellROX oxidative stress assay measured the level of oxidative stress. FIG. 6C: Expression of oxidative stress protective-related genes in hiPSC-ECs as determined by qPCR analysis. The hiPSC-ECs were treated with 5 μM Δ⁹-THC, 10 μM genistein, or their combination for 48 hours, and normalized to GAPDH. FIG. 6D: Genistein blocked monocyte adhesion in hiPSC-ECs treated with Δ⁹-THC. hiPSC-ECs were treated with 5 μM Δ⁹-THC and 10 μM genistein or vehicle (control) for 48 hours. U937 cells adherence to hiPSC-ECs was visualized by fluorescence microscope (left panel) and the intensity was quantified (right panel). FIG. 6E: Genistein prevents Δ⁹-THC-induced NF-κB phosphorylation in hiPSC-ECs. Cells were treated with 5 μM Δ⁹-THC, 10 μM genistein, or their combination for 48 hours. Total cell lysates were subjected to western blot analysis. FIG. 6F: Genistein attenuated the effects of Δ⁹-THC on TLR4/NF-kb-related genes. Cells were treated with 5 μM Δ⁹-THC, 10 μM genistein, or a combination of both for 48 hours, and the mRNA expressions of TLR4/NF-kb-related genes were measured by qPCR analysis. The levels of mRNA were normalized to GAPDH. FIG. 6G: Genistein prevented Δ⁹-THC-mediated inflammation-related genes in hiPSC-ECs. FIG. 6H: Genistein attenuates Δ⁹-THC-induced inflammation in hiPSC-ECs. The cells were treated with 5 μM Δ⁹-THC and 10 μM genistein for 48 hours and then replaced with fresh cell culture medium. Total RNA was isolated from hiPSC-ECs every other day. The mRNA expression of inflammation-related genes was quantified by qPCR and normalized to GAPDH. FIG. 6I: Genistein attenuated Δ⁹-THC-induced inflammation in hiPSC-ECs. The cells were treated with 5 μM Δ⁹-THC for 48 hours, then replaced with fresh cell culture medium, and 10 μM genistein was added. Total RNA was isolated from hiPSC-ECs every other day. The mRNA expression of inflammation-related genes was quantified by qPCR and normalized to GAPDH.

FIG. 7A to 7F illustrate using animal experiments to prove the response caused by genistein in vivo treatment of Δ⁹-THC according to Example 8. FIG. 7A: Isometric tension recordings of isolated mice thoracic aortas were performed using a wire myograph. Vascular concentration-dependent relaxation was induced by acetylcholine (ACh) in pre-constricted mouse thoracic arteries. FIG. 7B: The mRNA expression of inflammation-related genes in C57BL/6J mice were treated with (1) vehicle control (n=5/group), (2) Δ⁹-THC (n=5/group, Δ⁹-THC 1 mg/kg), and (3) Δ⁹-THC plus genistein (n=5/group, Δ⁹-THC 1 mg/kg plus genistein 50 mg/kg) (upper panel). The mRNA expression of inflammation-related genes in C57BL/6J mice were treated with (1) vehicle control (n=8/group), (2) Δ⁹-THC (n=8/group, Δ⁹-THC 1 mg/kg), (3) Δ⁹-THC plus genistein (n=8/group, Δ⁹-THC 1 mg/kg plus genistein 50 mg/kg), and (4) Δ⁹-THC plus rimonabant (n=3/group, Δ⁹-THC 1 mg/kg plus rimonabant 3 mg/kg) (bottom panel). FIG. 7C: Luminex analysis of inflammatory cytokines in plasma of C57BL/6J mice treated with vehicle control, Δ⁹-THC, or Δ⁹-THC plus genistein. FIG. 7D: The mRNA expression of oxidative stress protective-related genes in thoracic artery tissues from mice. FIG. 7E: Effect of Δ⁹-THC and genistein on NF-κB phosphorylation in mouse thoracic artery. Total cell lysates were prepared, and the expressions of phosphor-NF-κB were analyzed by ELISA (upper left). Superoxide dismutase (SOD) activity of serum from mouse (bottom left). The effect of Δ⁹-THC and genistein on NF-κB phosphorylation in mouse thoracic artery tissues (n=5). Total cell lysates were prepared, and the expressions of NF-κB, phosphorylated NF-κB, and β-actin were analyzed by western blot analysis (right panel). FIG. 7F: The levels reduced glutathione (GSH), Oxidized glutathione (GSSG), Total glutathione (T-GSH) and GSH/GSSG in the serum samples of mice were detected. Plasma isolated from C57BL/6J mice (n=5) treated with vehicle control, Δ⁹-THC, genistein, or their combination every day for 30 days was analyzed by the glutathione colorimetric assay kit (BioVision, K261).

FIG. 8A to 8C illustrate the effects of genistein and Δ⁹-THC on cardiac contraction in vivo or in vitro experiments according to Example 9. FIG. 8A: Engineered heart tissues (EHTs) were incubated in (1) vehicle, (2) 5 μM Δ⁹-THC, or (3) 5 μM Δ⁹-THC and 10 μM genistein and assayed for 48 hours. After treatment exposure, the contractility, relaxation and peak force of EHT were measured using the Sony imaging platform. FIG. 8B: Same as the former, analysis of heart rate and contraction-relaxation (CR) interval (48 hours). FIG. 8C: C57BL/6J mice were treated with vehicle control, Δ⁹-THC (1 mg/kg i.p.); genistein (50 mg/kg p.o.): Δ⁹-THC (1 mg/kg i.p.) and genistein (50 mg/kg p.o.); rimonabant (3 mg/kg p.o.); or rimonabant (3 mg/kg p.o.) and Δ⁹-THC (1 mg/kg i.p.). Mice were anesthetized with 1%-2% isoflurane, and images were acquired in the parasternal long-axis (PLA) and parasternal short axis (PSA) on a Visualsonic Vevo 2100 platform. The ventricular (LV) dimensions were traced offline, and the ejection fraction (EF) was calculated using Simpson's biplane method of discs.

FIG. 9A to 9E illustrate the effect of Δ⁹-THC and genistein on the formation of chronic atherosclerosis according to Example 10. FIG. 9A: The body weights of Ldlr^{-/-} mice after exposure to (1) vehicle control (n=10), (2) Δ⁹-THC (n=12), or (3) Δ⁹-THC plus genistein (n=12). FIG. 9B: Lipid profiles of serum samples (left panel) and blood pressure (right panel) of Ldlr^{-/-} mice after exposure to (1) vehicle control (n=10 (left), n=3 (right)), (2) Δ⁹-THC (n=12 (left), n=4 (right)), and (3) Δ⁹-THC plus genistein (n=12 (left), n=4 (right)). The blood pressure was measured by tail-cuff. FIG. 9C: Oil red O staining of atherosclerotic plaques in cross-sections at the aortic root level (upper panel). Quantitation of atherosclerotic plaques: (1) vehicle control (n=10), (2) Δ⁹-THC (n=10), and (3) Δ⁹-THC plus genistein (n=11) (bottom panel). FIG. 9D: Gross images of oil red O stained Ldlr^{-/-} thoracic aorta are shown (left panel). Quantification of plaque size by en face analysis of thoracic aorta after exposure to (1) vehicle control (n=10), (2) Δ⁹-THC (n=12), or (3) Δ⁹-THC plus genistein (n=12). FIG. 9E: Immunostaining of CD68 in cross-sections at the aortic root level (upper panel). Quantitation of CD68-positive area from (1) vehicle control (n=10), (2) Δ⁹-THC (n=9), and (3) Δ⁹-THC plus genistein (n=10) (bottom panel).

FIG. 10A to 10C illustrate the effects of Δ⁹-THC and genistein on the treatment of atherosclerosis in vivo using the Apoe^{-/-} mouse model according to Example 10. FIG. 10A: Gross images of carotid arteries after oil red O staining are shown (left panel). The scale bar represents 1 mm. A diagram of partial ligation of the carotid artery is shown (right panel). The left common carotid artery (LCA), external carotid artery (ECA), internal carotid artery (ICA), and superior thyroid artery (STA) were ligated, leaving the occipital artery (OA) open. The right subclavian artery (RSA), right common carotid artery (RCA), and left subclavian artery (LSA) were unligated and remained patent. FIG. 10B: Carotid artery sections were counterstained with hematoxylin and eosin (H&E), and a representative slide was presented with scale bars at 100 μm (left panel). Oil red O staining of atherosclerotic plaques in cross-section of mouse carotid artery with scale bar at 100 μm. The atherosclerotic plaques were quantified (right panel). FIG. 10C: Carotid artery sections were immunostained with an anti-F4/80 antibody. High-magnification images of the green-boxed area are shown with the scale bar at 250 mm (top left). Quantification of F4/80-positive area within carotid artery sections (top right). Carotid artery sections were immunostained with an anti-CD68 antibody. High-magnification images of the green-boxed area are shown with the scale bar at 250 mm (bottom left). Quantification of CD68-positive area within carotid artery sections is shown (bottom right).

FIG. 11A to 11D illustrate using fluorescently labeled genistein to detect the binding situation in vivo and in vitro according to Example 11. FIG. 11A: The cellular distribution of BODIPY517/547-genistein in hiPSC-ECs. The cells were treated with BODIPY517/547-genistein (5 μM) for 6 or 12 hours and subjected to fluorescence microscopy for BODIPY517/547-genistein (red fluorescence) and DAPI (blue fluorescence). High-magnification images of the green-boxed area are shown with the scale bar at 100 mm. FIG. 11B: BODIPY-genistein attenuated NF-κB phosphorylation induced by Δ⁹-THC in mouse thoracic artery. Total cell lysates were prepared, and the expression of phosphorylated NF-κB and NF-κB was analyzed by western blot analysis. Immunoblots were quantified by ImageJ software (left panel). FIG. 11C: The mRNA expression of inflammation-related genes and oxidative stress protective-related genes in thoracic artery tissues from mice is shown after normalizing to GAPDH (upper panel). Quantitative analysis results of SOD, GSH, GSSG, T-GSH and GSH/GSSG in mouse serum after treatment (bottom panel). FIG. 11D: Adult male BALB/c nude mice (n=5) were intravenously injected with BODIPY517/547-genistein at a dose of 5 mg/kg. The in vivo fluorescence images were detected by the IVIS Spectrum at indicated time points. All mice were sacrificed at 48 hours, and major organs were collected and then fixed using formalin (left panel). Fluorescence images were quantified using the IVIS imaging system. The fluorescence intensity peaked at 24 hours. The fluorescence images of BODIPY517/547-genistein were quantified by ImageJ software (middle upper). Images of the brain, heart and lung, thoracic aorta, liver, spleen, kidney, intestine, colon, and blood were obtained from mice (n=5) (right panel). Fluorescence images were quantified using the ImageJ software (middle bottom).

FIG. 12A to 12C illustrate the co-localization of genistein and CB1 receptors in vivo by confocal microscopy according to Example 11. FIG. 12A: En face immunofluorescence staining was performed to observe the localization of BODIPY517/547-genistein and CB1 receptors in vascular endothelial cells from the mouse thoracic aorta. Mice were either injected with BODIPY517/547-genistein alone or pretreated with 100 nM rimonabant for 30 min prior to injection of BODIPY517/547-genistein. Mouse aortas were isolated and permeabilized with Triton X100. The aortas were incubated with anti-CB1 antibody Alexa Fluor 488. Fluorescent z stack images were collected at 0.5 μm steps by laser scan confocal microscopy. En face immunofluorescence images of BODIPY517/547-genistein (red fluorescence) and CB1 receptor (Alexa Fluor 488, green fluorescence) in vascular endothelial cells after treatment with BODIPY517/547-genistein for 48 hours. Scale bar, 20 μm. FIG. 12B: Scatter plots of co-localization analysis in vascular endothelial cells from mouse thoracic aorta at 48 hours. The x axis represents CB1 receptors (Alexa Fluor 488, green fluorescence), and the y axis represents BODIPY517/547-genistein (red fluorescence). FIG. 12C: Quantification of BODIPY517/547-genistein co-localized with CB1 receptors. The co-localized signal is colored orange.

FIG. 13 illustrate the effect of genistein on the neurobehavioral effects of Δ⁹-THC according to Example 12. C57BL/6J mice were exposed to (1) vehicle control (intraperitoneal [i.p.], and oral [p.o.], n=8), (2) Δ⁹-THC at 20 mg/kg i.p. and p.o. vehicle (n=9), (3) genistein at 50 mg/kg p.o. and i.p. vehicle (n=8), and (4) combination of Δ⁹-THC at 20 mg/kg i.p. and genistein at 50 mg/kg p.o. (n=9). Spontaneous activity was assessed by the total distance moved in an activity chamber and was reduced with Δ⁹-THC treatment. Hypothermia was assessed by measuring body temperature using a rectal probe before and 60 min after treatment. The catalepsy test measures the time a mouse remained immobile on the bar. Analgesia was measured by placing mice on a hot plate and recording the time for the mouse to withdraw or lick.

About the above schema: error bars represent mean±SEM. * or # represent p<0.05: ** or ## represent p<0.01: *** or ### represent p<0.001: **** or #### represent p<0.0001: ns represent no significant versus vehicle. The retention time (TR) means the time required for the recovery of mRNA basal level.

### DETAILED DESCRIPTION OF THE INVENTION

A detailed description and the technical contents of the present invention are given below with reference to the accompanying drawings. Furthermore, for easier illustrating, the drawings of the present invention are not a certainly the practical proportion and are not limited to the scope of the present invention.

Unless defined otherwise, all technical and scientific terms used herein have the same meaning as commonly understood to one of ordinary skill in the art to which this disclosure belongs. As used throughout the instant application, the following terms shall have the following meanings.

The use of “or” means “and/or” unless stated otherwise. The use of “comprise” means not excluding the presence or addition of one or more other components, steps, operations, or elements to the described components, steps, operations, or elements, respectively. Similarly, “comprise,” “comprises,” “comprising” “include,” “includes,” and “including” are interchangeable and not intended to be limiting. As used herein and in the appended claims, the singular forms “a,” “an,” and “the” include plural referents unless the context dictates otherwise. The terms “a”, “an,” “the,” “one or more,” and “at least one,” for example, can be used interchangeably herein.

The present invention provides a use of genistein and its phosphorylated form (that is, the phosphate ester derivative produced after phosphorylation), which is used for the preparation of a medicament for treatment or prevention of diseases mediated by cannabinoid receptor type 1 (CB1 receptor) in an individual. The structure of the genistein is as follows.

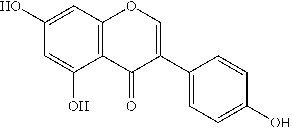 [SMILES: O=c1c(-c2ccc(O)cc2)coc2cc(O)cc(O)c12]

Specifically, the use of genistein phosphate ester derivatives effectively improves water solubility and bioavailability, thereby enhancing the pharmacological benefits of genistein when administered to individuals. Further details can be found in Taiwan Patent Publication No. I756495B.

The term “subject” refers to mammals including, but not limited to, humans, non-human primates, sheep, dogs, rodents (e.g., mouse, rat, etc.), guinea pigs, cats, rabbits, cows, horses, and non-mammals including chickens, amphibians, and reptiles. Preferably, the subject is human.

The term “treatment” as used herein is defined as maintaining or reducing the symptoms of a pre-existing condition when administered genistein or its phosphate ester derivative conjugate compared to the same symptom in the subject prior to the administration of genistein or its phosphate ester derivative conjugate to the subject. The term “prevention” is defined as delaying the onset of a CB1-mediated disease in an individual.

The term “CB1 receptor-mediated diseases” as used herein includes cardiovascular diseases, mental disorders, anxiety disorders, depression, epilepsy, neurodegenerative diseases, cognitive impairment, brain trauma, glaucoma, Parkinson's disease, Alzheimer's disease, Huntington's chorea, tremors, senile dementia, Tourette syndrome, cancer, hypotonia, septic shock, hypotension, multiple sclerosis, vomiting, asthma, eating disorders, obesity, diabetes, diseases associated with demyelination, neuroinflammation, viral encephalitis, liver cirrhosis, or gastrointestinal diseases.

Specifically, said treatment or prevention of CB1 receptor-mediated diseases in the individual involves alleviating oxidative stress and endothelial dysfunction induced by Δ⁹-tetrahydrocannabinol, dronabinol, or nabilone. Further, said oxidative stress involves a decrease in the expression levels of antioxidant genes and an increase in the expression levels of reactive oxygen species (ROS)-related genes, wherein the antioxidant genes include superoxide dismutase 1 (SOD1) gene, superoxide dismutase 2 (SOD2) gene, catalase (CAT) gene, or glutathione peroxidase 1 (GPX1) gene, and the ROS-related genes involve the expression of NADPH oxidase 1 (NOX1) or inducible nitric oxide synthase (NOS2).

In some embodiment, said treatment or prevention of CB1 receptor-mediated diseases in the individual involves alleviates inflammation induced by Δ⁹-tetrahydrocannabinol, dronabinol, or nabilone. Specifically, the medicament reduces mRNA expression levels of pro-inflammatory cytokines and chemokines induced by Δ⁹-tetrahydrocannabinol, dronabinol, or nabilone.

The term “cellular inflammatory cytokines” as used herein refers to cytokines that promote inflammatory responses in cells. Common cellular inflammatory cytokines include interleukin-1 (IL-1), interleukin-6 (IL-6), and tumor necrosis factor (TNF). The term “chemotactic factors” can also be referred to as chemotactic hormones, chemotaxins, or chemokines, which are signaling proteins secreted by cells. The main function of chemotactic factors is to induce the migration of cells, whereby cells move towards the source of the chemotactic factor in response to its increasing concentration.

Another aspect of the present invention provides a pharmaceutical composition for treatment or prevention of CB1 receptor-mediated diseases, comprising an effective amount of genistein and its phosphate ester derivative. The phosphate ester derivative of genistein is genistein 7-O-phosphate (G7P) of the following formula (I), or genistein 4′-O-phosphate (G4′P) of the following formula (II). Specifically, the phosphate ester derivative of genistein can effectively improve the water solubility and bioavailability of genistein. In a preferred embodiment, genistein phosphate ester was biotransformed from isoflavone by Bacillus subtilis var. natto BCRC 80517 (Bioresource Collection and Research Center, Taiwan), and the preparation method can be found in Hsu et al., Food Research International 53 (2013) 487-495.

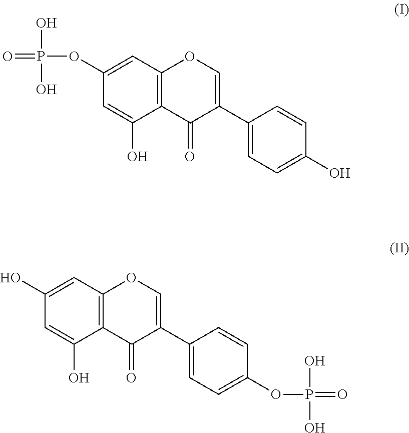 [SMILES: O=c1c(-c2ccc(O)cc2)coc2cc(OP(=O)(O)O)cc(O)c12 ; O=c1c(-c2ccc(OP(=O)(O)O)cc2)coc2cc(O)cc(O)c12]

The phosphorylation stated herein refers to the addition of a phosphate group to a protein or another type of molecule. This reaction plays an important role in energy metabolism and signal transduction in a living body and is a critical to biochemistry. Currently known phosphorylation entails kinase (which is a phophotransferase) or phosphorylase, both falling within the EC 2.7 category, and the reaction requires ATP as the source of energy and Mg²⁺ ions as a cofactor. Generally, the aforesaid enzymes hydrolyze ATP and transfer γ-phosphate to the substrate. Protein kinase is the most common large-molecule phosphorylation enzyme, is responsible for modifying, through phosphorylation, a wide range of proteins with different functions, and is an essential means for regulating signal transduction in a living body. More and more physiological phenomena, such as whether an enzyme is activated or not, have been found to be related to the phosphorylation or dephosphorylation of protein. Certain amino acid sites on a protein molecule, such as the —OH functional group of serine, threonine, or tyrosine, or the imidazole ring of histidine, can be modified by protein kinase through phosphorylation such that the molecule is activated by the addition of phosphoric acid, and this phosphoric acid can be subsequently removed with protein phosphatase to render the molecule deactivated. There are also many examples in which similar reactions produce the opposite effects. As to small-molecule phosphorylation enzymes such as acetokinase, glycerol kinase, arginine kinase, shikimate kinase, mevalonate kinase, and nucleoside kinase, they are responsible for such crucial catalytic reactions in the metabolic pathways in a living body as glycolysis, the biosynthesis of amino acids, the biosynthesis of cholesterol, and the biosynthesis of nucleotides. Phosphorylation enzymes can also be divided by the source of the phosphate group into the following two types. The first type uses a phosphoric acid monoester as the phosphate donor and is generally capable of hydrolyzing ATP and transferring γ-phosphate to the substrate. The second type uses a phosphonate diester or pyrophosphate as the phosphate donor instead.

In a preferred embodiment, the phosphate ester derivative of genistein is obtained by microbial fermentation of genistein or by contacting or culturing genistein with a polyphenolic flavonoid phosphate synthase.

In some embodiments, the microorganism comprises a nucleic acid sequence encoding (or expressing) a polypeptide having a homologous protein sequence with a similarity of at least 70% to the polyphenolic flavonoid phosphate synthase (SEQ ID NO: 1), said polypeptide comprising a conserved region, said conserved region being based on SEQ ID NO: 1 and sequentially comprising: an ATP binding region, including active catalytic sites of lysine 27 (Lys27), arginine 102 (Arg102), and glutamic acid 282 (Glu282): a substrate binding region, including a conserved motif DDHHFYIDAMLDAKAR (SEQ ID NO: 2), and active catalytic sites of aspartic acid 627 (Asp627), histidine 629 (His629), and histidine 630 (His630); and a phosphorylated histidine catalytic region, including an active catalytic site of histidine 795 (His795).

The term “microorganism” as used herein refers to tiny organisms that are not easily visible to the naked eye. This includes microorganisms with cellular structures such as bacteria, fungi, actinomycetes, protozoa, algae, and other microorganisms. It also includes organisms without complete cellular structures such as viruses, rickettsiae, and chlamydiae. The above-mentioned microorganism can normally express the nucleic acid sequence after transfer or transformation of the genetic material of the above-mentioned polypeptide (sequentially comprising the amino acid sequence of the ATP-binding domain, the substrate-binding domain and the phosphorylated histidine catalytic domain) through genetic engineering or molecular biotechnology.

The aforementioned microorganism may be a microorganism that has been genetically modified to express the aforementioned polypeptide, which may include genetic modification of the organism to enhance or enhance the production of the polypeptide in the organism. A genetically modified microorganism may be a genetically modified bacterium, unicellular organisms, microalgae, fungi, etc. This genetically modified microorganism has a genome modified (i.e. mutated or altered) from its normal form (i.e. wild-type or naturally occurring) such that the desired result can be achieved. Genetic modification of microorganisms can be accomplished using typical strain development and/or molecular genetic techniques. Such techniques are known in the art and are generally disclosed for use with microorganisms. A genetically modified microorganism may be a microorganism in which a nucleic acid molecule has been inserted, deleted, or modified (i.e. mutated; such as by nucleotide insertions, deletions, substitutions, and/or inversions) in such a way that this kind of modifications can provide the desired effect on the microorganism.

The term “sequence” refers to the nitrogenous bases in a nucleic acid structure, of which there are four types: A (adenine), T (thymine), C (cytosine), and G (guanine). These bases can be linearly arranged in infinite combinations to express genes, and each gene has a unique base sequence.

The terms “polypeptide,” “polypeptide fragment,” “peptide,” and “protein” are used interchangeably herein to refer to polymers of amino acid residues, and variants and synthetic analogs thereof. Accordingly, these terms apply to amino acid polymers in which one or more amino acid residues are synthetic non-naturally occurring amino acids, such as chemical analogs corresponding to naturally occurring amino acids, and polymers suitable for naturally occurring amino acids. In certain aspects, polypeptides can include enzymatic polypeptides (or “enzymes”), which typically catalyze (i.e. increasing the rate of) various chemical reactions.

The terms “Conserved” as used herein refers to the situation in biology that is similar or identical within a nucleic acid sequence, protein sequence, protein structure or polysaccharide sequence: it may occur between species, or between different molecules arising from the same organism. From an evolutionary point of view, it means a state in which a particular sequence continues to be preserved during the process of speciation.

As used herein, “identity” of an amino acid sequence refers to the degree to which two sequences are mutually indistinguishable, and “similarity” refers to the same ratio and/or retention ratio between the two sequences. Those of ordinary skill in the art to which the present invention pertains should understand that the long-chain amino acids of polypeptides and proteins are only partially functional in their amino acid sequences, which are called functional motifs. Proteins have the same function when they have the same functional motif; in general, when the amino acid sequence of a polypeptide or protein is at least 40% identical, it has the same function (refer to How Proteins Work, Williamson, 2011).

In a preferred embodiment, the pharmaceutical composition comprises at least one other therapeutic agent, wherein the at least one other therapeutic agent is anti-obesity agents, appetite suppressants, anti-diabetic agents, anti-hyperlipidemic agents, lipid-lowering agents, blood cholesterol-lowering agents, fat-modulating agents, cholesterol-lowering agents, fat-reducing agents, high-density lipoprotein-raising agents, anti-hypertensive agents, preparations for treating sleep disorders, preparations for treating substance abuse or addiction disorders, anti-anxiety agents, antidepressants, antipsychotic agents, cognitive enhancers, preparations for treating cognitive disorders, preparations for treating Alzheimer's disease, preparations for treating Parkinson's disease, anti-inflammatory agents, preparations for treating neurodegenerative disorders, preparations for treating arteriosclerosis, preparations for treating respiratory conditions, preparations for treating intestinal diseases, preparations for treating liver cirrhosis, or antineoplastic agents.

In some embodiments, the pharmaceutical composition is used for the treatment of chronic dependence, alcohol dependence, or drug abuse. Preferably, the pharmaceutical composition is used for the treatment of cannabis abuse.

In some embodiments, the pharmaceutical composition enhances the analgesic activity of analgesic drugs or anesthetic drugs.

### Embodiments

It should be understood that the examples and embodiments described herein are for illustrative purposes only, and that various modifications or changes therefrom will be suggested to those skilled in the art, and that such modifications or changes will be included within the spirit and scope of the application and within the scope of the attached claims. All publications, patents, and patent applications cited herein are hereby incorporated by reference in their entirety for all purposes.

### EXAMPLE 1

### Screening of CB1 Receptor Antagonists

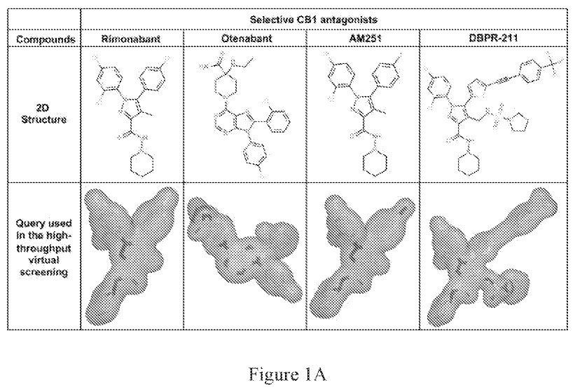

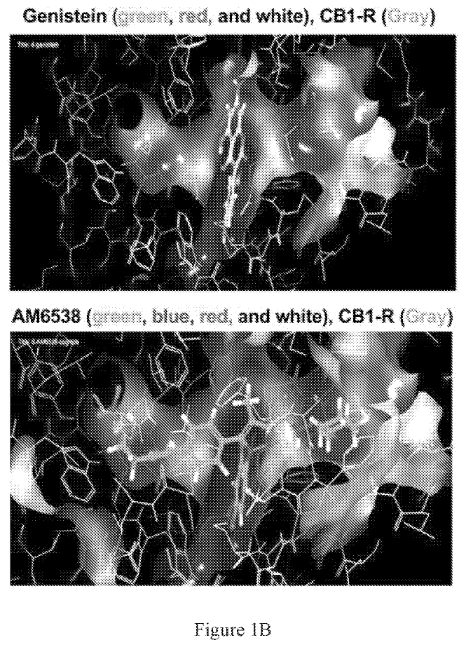

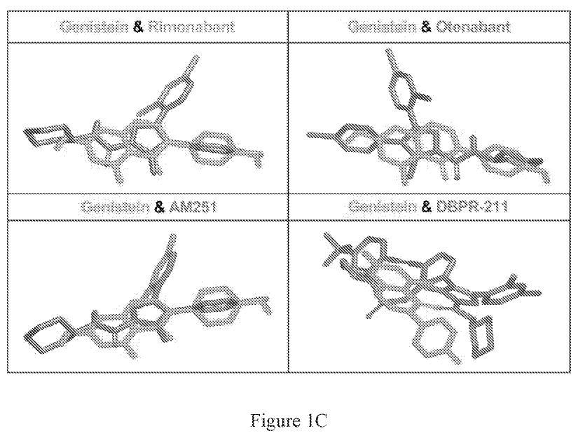

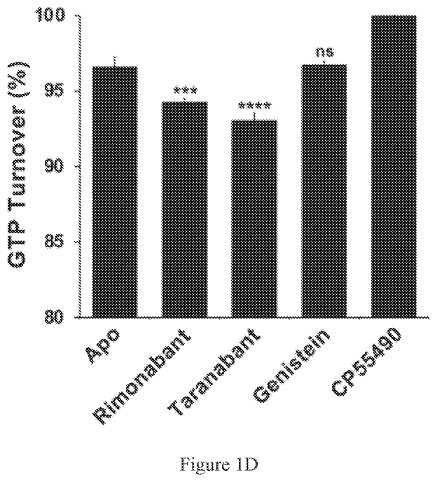

Previous studies have shown that cannabis use is associated with cardiovascular disease, and analysis based on the UK Biobank (UKB) confirmed that cannabis users showed a higher risk of myocardial infarction than non-cannabis users (DeFilippis et al., 2018). Based on this correlation, the inventors of the present invention further used the SWEETLEAD chemical database to screen four specific CB1 receptor antagonists (rimonabant, otenabant, AM251 and DBPR-211) using the ROCS software suite (as shown in FIG. 1A). In addition, using ligand-based high-throughput virtual screening technology and chemical databases, 62 chemical compounds that were structurally homologous with the 4 selective CB1 antagonists were found. For further screening, molecular docking analysis was performed to probe the interactions between the chemical compounds and the CB1 receptor with the selective CB1 antagonist AM6538 as a positive control. The results showed that genistein, an abundant isoflavone compound in beans, can bind to the CB1 receptor (as shown in FIG. 1B); and the shape of genistein is similar to the structure of the specific CB1 antagonist mentioned above (FIG. 1C). Furthermore, please refer to FIG. 1D, an in vitro GTPase assay found that genistein functions as a neutral antagonist. Moreover, radioligand binding assays for predicted targets of genistein binding revealed that genistein binds human CB1 with an IC₅₀ of 150 nM (not shown in the figure).

### EXAMPLE 2

### Cytotoxicity of Δ⁹-THC on Endothelial Cells

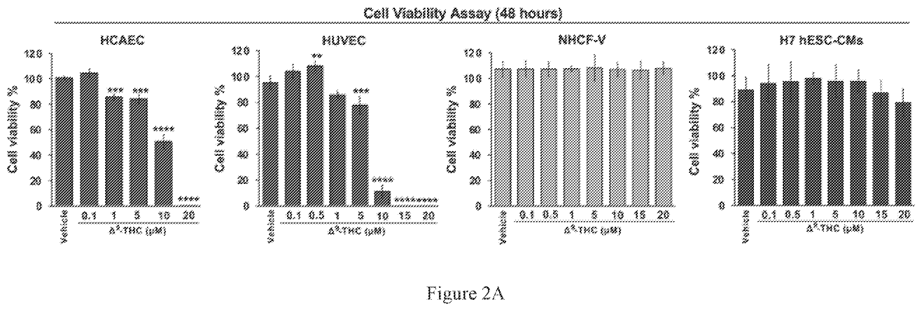

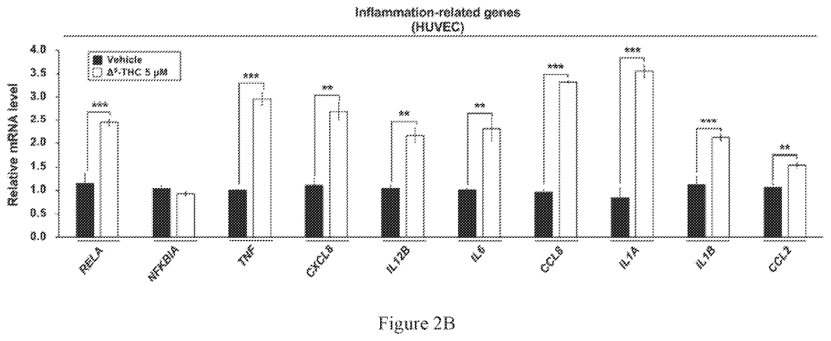

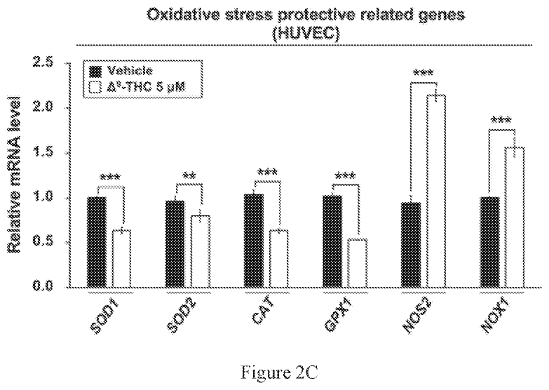

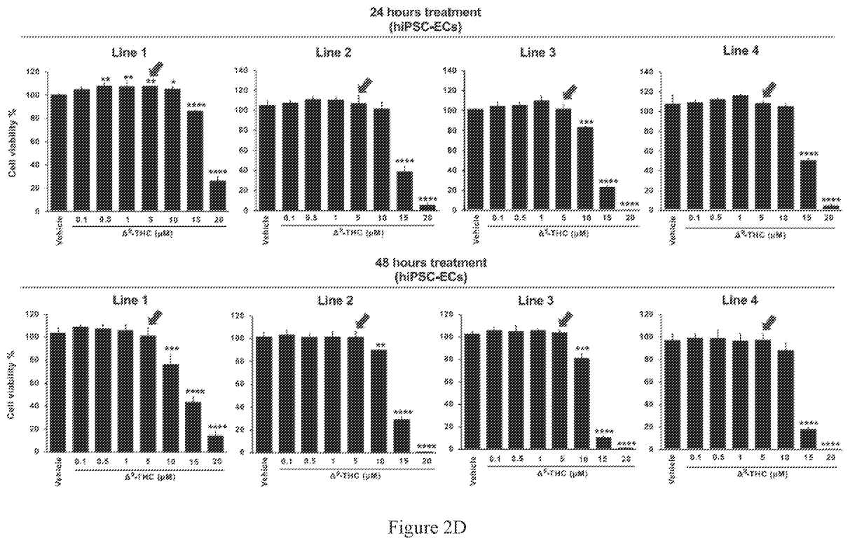

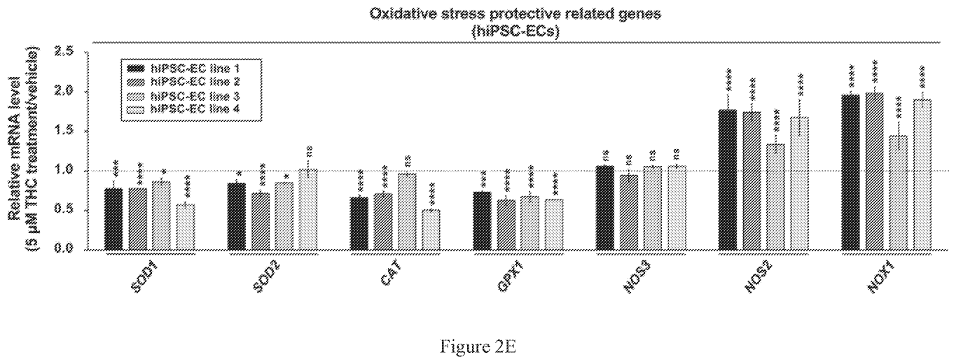

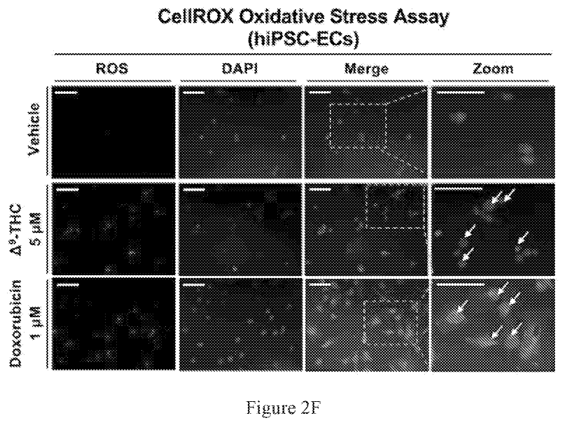

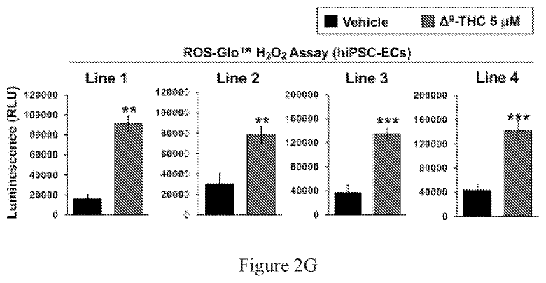

Next, since the psychoactive components of cannabis, Δ⁹-THC, dronabinol or nabilone, are thought to cause cardiovascular problems by binding to CB1 receptors, Δ9-THC was used to evaluate its cytotoxicity in three different cell types: (1) human endothelial cells (human umbilical vein endothelial cells [HUVECs] and human coronary artery endothelial cells [HCAECs]), (2) human embryonic stem cell-derived cardiomyocytes (H7 hESC-CMs), and (3) normal human cardiac fibroblasts-ventricular (NHCF-V) cells. As shown in FIG. 2A, Δ⁹-THC induced cytotoxicity in human endothelial cells without any adverse effect on cardiomyocytes or cardiac fibroblasts.

On the other hand, inflammation and oxidative stress are believed to cause endothelial dysfunction, so we next investigated the expression of these genes in HUVECs exposed to Δ⁹-THC to uncover the mechanism of such endothelial cytotoxicity. The results showed that Δ⁹-THC induced the expression of inflammation-related genes (FIG. 2B) and reduced the antioxidant-related gene expression (FIG. 2C). HUVECs were derived from large pools of female patients with environmental exposures that might affect the phenotype with Δ⁹-THC. However, hiPSC-ECs are free of previous environmental exposures. Therefore, hiPSC-EC lines were generated from 4 healthy individuals through a chemically defined differentiation protocol, and found that Δ⁹-THC also induced cytotoxicity (FIG. 2D). Accordingly, it was confirmed that Δ⁹-THC induces cytotoxicity in human endothelial cells.

### EXAMPLE 3

### Cytotoxicity of Δ⁹-THC on hiPSC-ECs

Since antioxidant-related genes were reduced by Δ⁹-THC treatment in HUVECs (FIG. 2C), the expression of these genes in hiPSC-ECs was further evaluated. The results showed that Δ⁹-THC also decreased mRNA expression of antioxidant-related genes, including superoxide dismutase 1 (SOD1), superoxide dismutase 2 (SOD2), catalase (CAT), and glutathione peroxidase 1 (GPX1) in hiPSC-ECs. In addition, ROS-related genes implicated in endothelial dysfunction, such as NADPH oxidase 1 (NOX1) and inducible nitric oxide synthase (NOS2), were upregulated in hiPSC-ECs after Δ⁹-THC treatment (FIG. 2E). The result of immunofluorescence staining of CellROX oxidative stress assay showed that Δ⁹-THC treatment of hiPSC-ECs also induced cellular oxidative stress (FIG. 2F) with increased hydrogen peroxide (H₂O₂) levels (FIG. 2G).

### EXAMPLE 4

### Effect of Δ⁹-THC on the Inflammatory Response of hiPSC-ECs

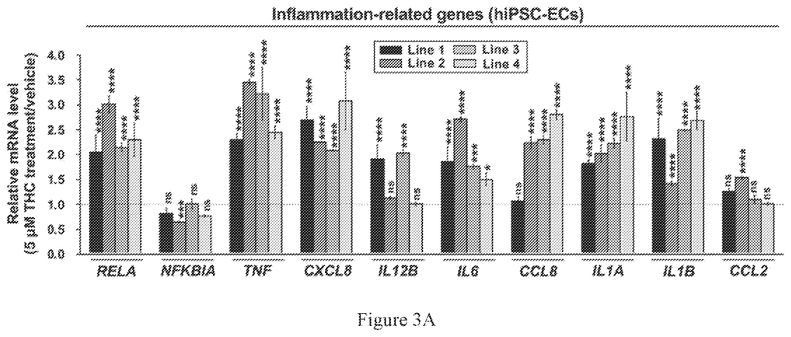

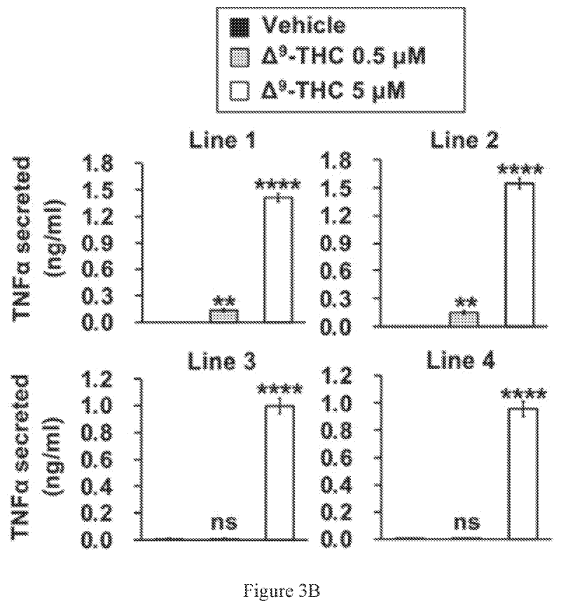

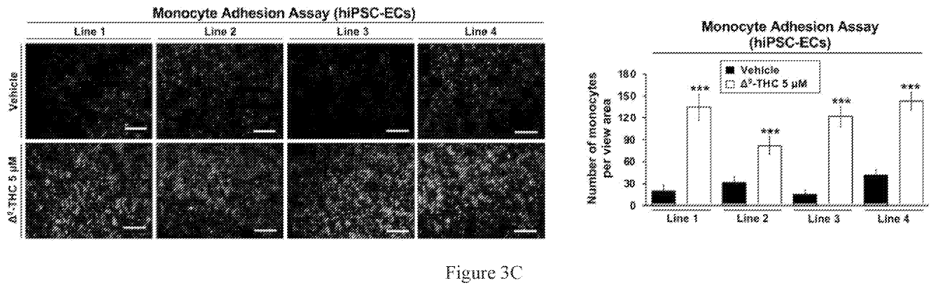

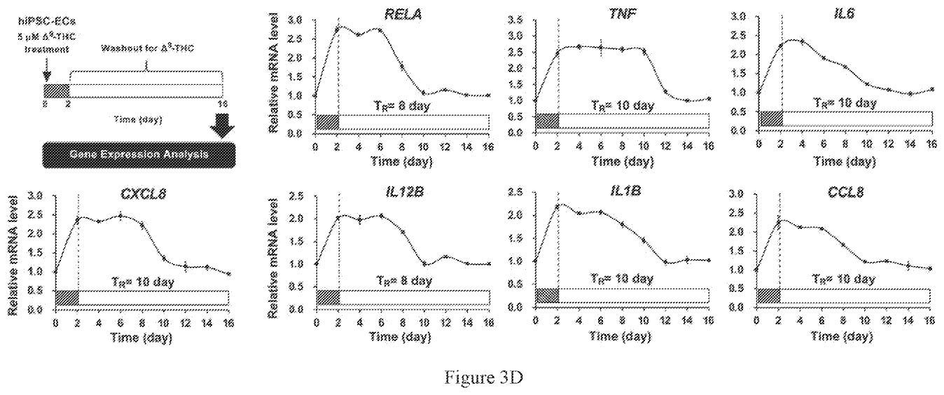

Numerous previous studies have pointed to the anti-inflammatory effects of cannabis and Δ⁹-THC. The present invention next studying the gene expression related to inflammation in hiPSC-ECs treated with Δ⁹-THC. The result showed that proinflammatory cytokines and chemokines were increased in response to Δ⁹-THC, while I kappa B (NFKBIA), a specific inhibitor of the NF-κB transcription factor, was inhibited by Δ⁹-THC (FIG. 3A). In addition, after Δ⁹-THC treatment, TNF-α concentration was increased in the cell culture medium of hiPSC-ECs (FIG. 3B). Moreover, by detecting the adhesion of monocytes to hiPSC-ECs after Δ⁹-THC treatment, it was found that monocytes had increased adhesion to hiPSC-ECs (FIG. 3C).

The longitudinal effects of Δ⁹-THC-induced inflammation were assessed in hiPSC-ECs using wash-out experiments. After treatment with Δ⁹-THC for 48 hours, the supernatant in hiPSC-ECs was replaced with fresh medium. The expression of inflammation-related genes in hiPSC-ECs was evaluated every other day for 2 weeks. The Δ⁹-THC-induced expressions of proinflammatory cytokines and chemokines were sustained for 8 to 10 days after the initial exposure (FIG. 3D). Thus, Δ⁹-THC-induced inflammation persisted four to five times longer than the initial exposure.

### EXAMPLE 5

### Mechanism of Oxidative Stress Induced by Δ⁹-THC

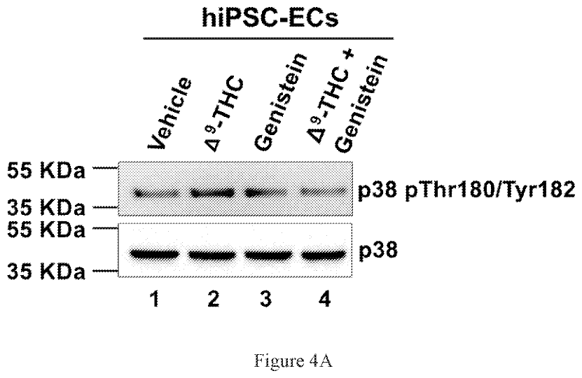

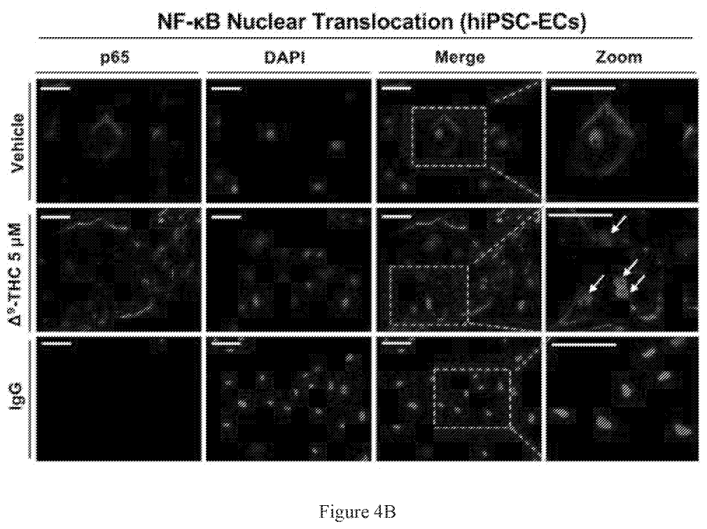

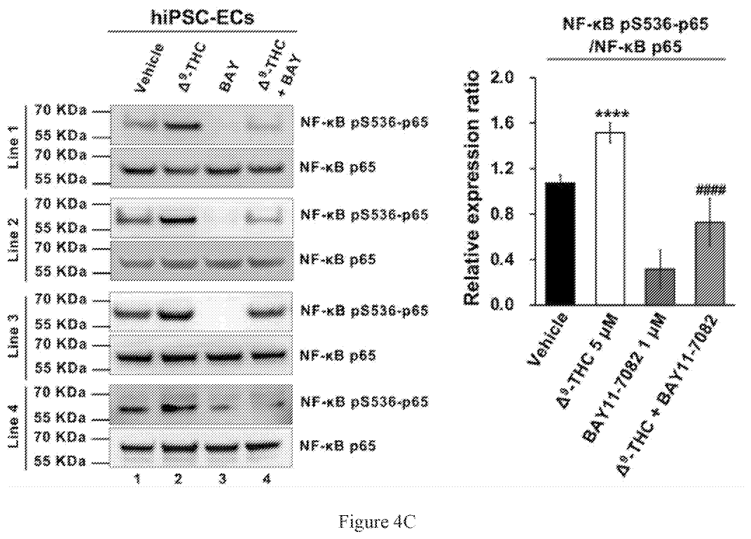

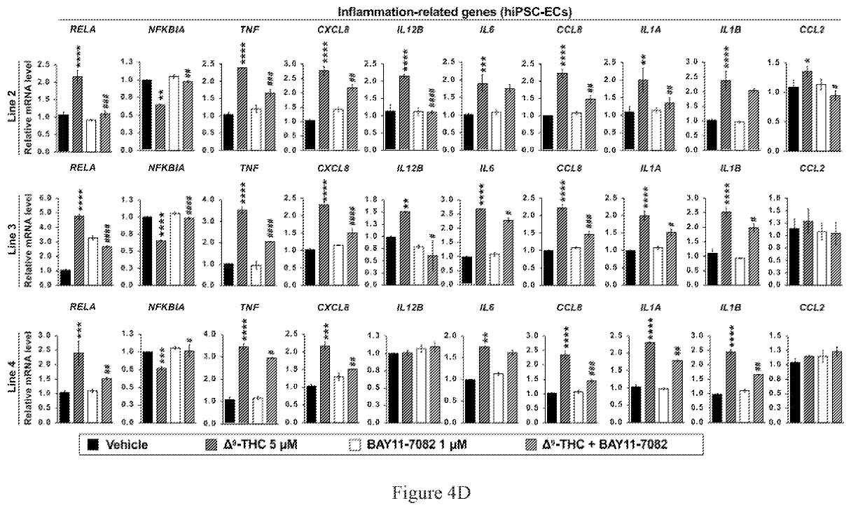

Herein, the inventor sought to elucidate the mechanisms of how Δ⁹-THC causes oxidative stress in the vasculature via CB1. The elevated levels of TNF-α suggested that NF-κB could cause activation of inflammatory genes and promote oxidative stress via the nuclear translocation of the p65 component of the complex. The Δ⁹-THC-binding activates the mitogen-activated protein kinase (MAPK) pathway. In hiPSC-ECs, Δ⁹-THC also causes increased phosphorylation of p38 (FIG. 4A). Thus, the cellular distribution of NF-κB was subsequently investigated in hiPSC-ECs after Δ⁹-THC treatment. Immunofluorescent staining revealed that Δ⁹-THC induced NF-κβ's nuclear translocation of NF-κB (FIG. 4B) and phosphorylation of NF-κB in hiPSC-ECs. The Δ⁹-THC-mediated phosphorylation of NF-κB was abrogated by the selective NF-κB inhibitor BAY11-7082 (FIGS. 4C and 4D). Based on the above experimental results, the mechanism of Δ⁹-THC causing oxidative stress is related to its activation of NF-κβ in hiPSC-ECs.

### EXAMPLE 6

### Effect of CB1 Receptor on the Action of Δ⁹-THC and Genistein

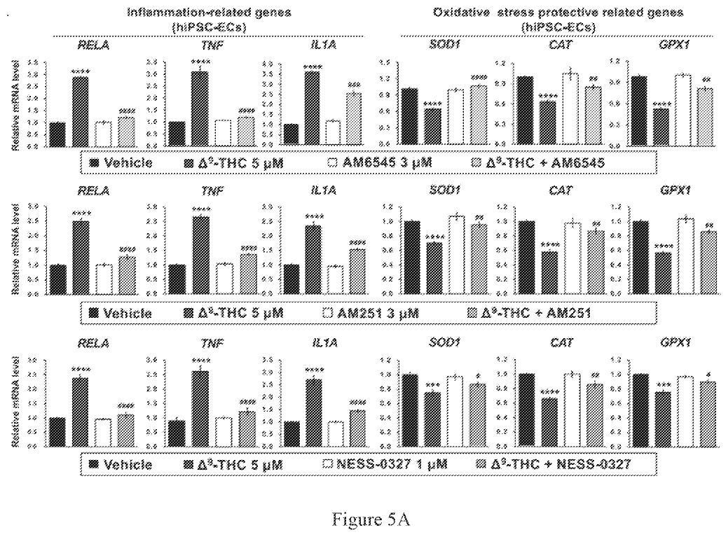

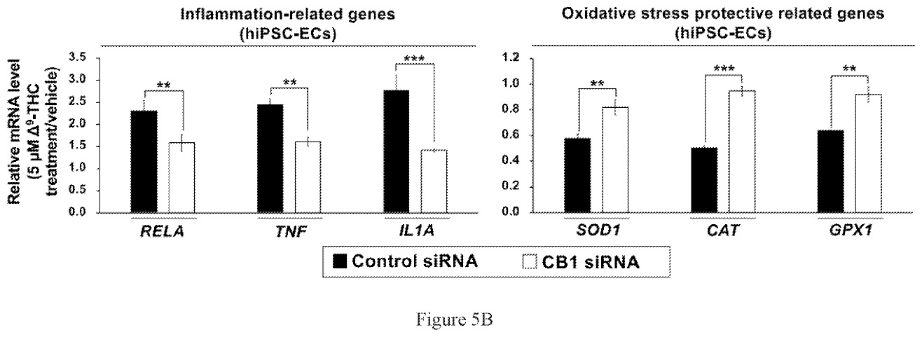

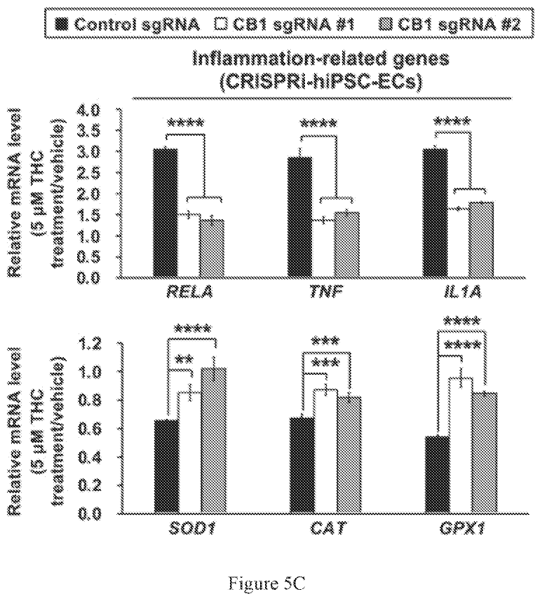

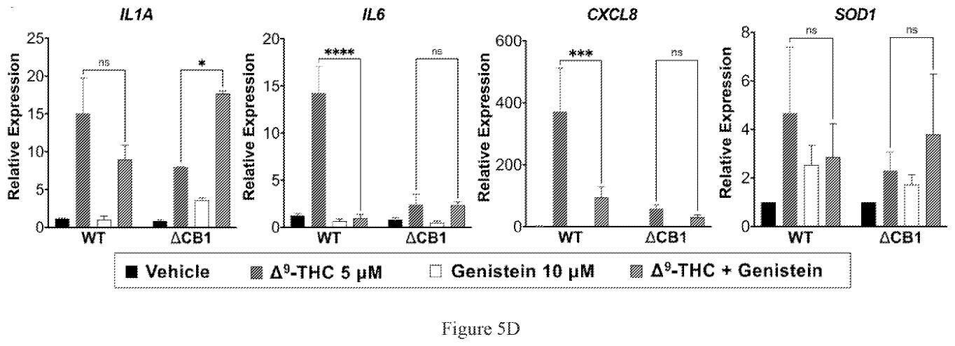

The CB1 receptor is implicated in the pathological effects of Δ⁹-THC on the vasculature. The inventor next employed pharmacologic and genetic inactivation of the CB1 receptor to determine whether the CB1 receptor plays a role in Δ⁹-THC-induced effects in hiPSC-ECs. The selective CB1 antagonists AM6545, AM251, and NESS-0327 reversed Δ⁹-THC-induced mRNA expression of inflammation-related genes and oxidative stress protective-related genes (FIG. 5A). The Δ⁹-THC-induced effects were also reversed by the knockdown of CB1 by siRNA (FIG. 5B).

Similarly, the inventor also found that CRISPR interference (CRISPRi) of CB1 expression reversed Δ⁹-THC-induced inflammation and oxidative stress in hiPSC-ECs (FIG. 5C).

On the other hand, Genistein is known to ameliorate TNF-α-mediated inflammation and oxidative stress in endothelial cells. Therefore, the inventor tested if the CB1 receptor was required for genistein to block the effects of TNF-α in hiPSC-ECs using CRISPRi to assess CB1 expression. The result showed that the CB1 receptor was required for genistein to attenuate the inflammatory effects of TNF-α (FIG. 5D).

### EXAMPLE 7

### Effects of Genistein on the Responses Induced by Δ⁹-THC

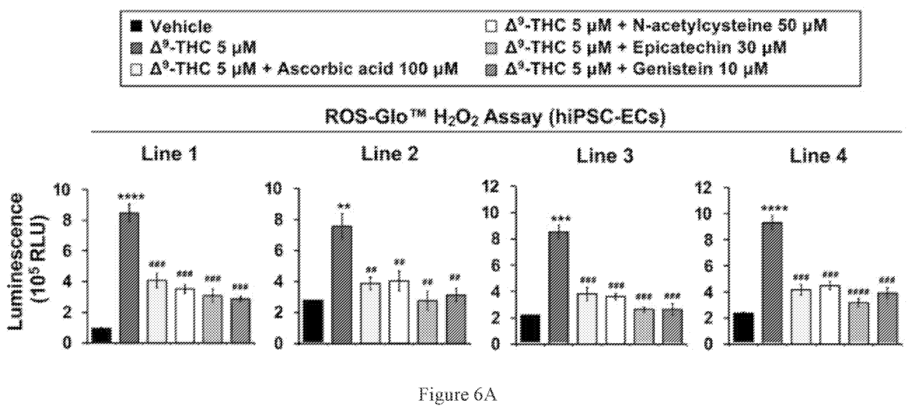

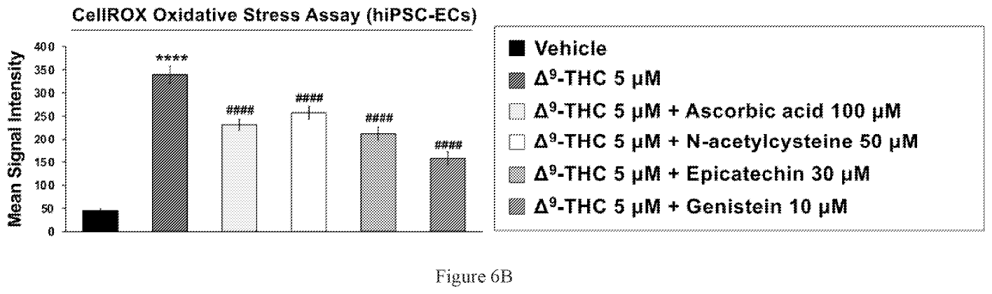

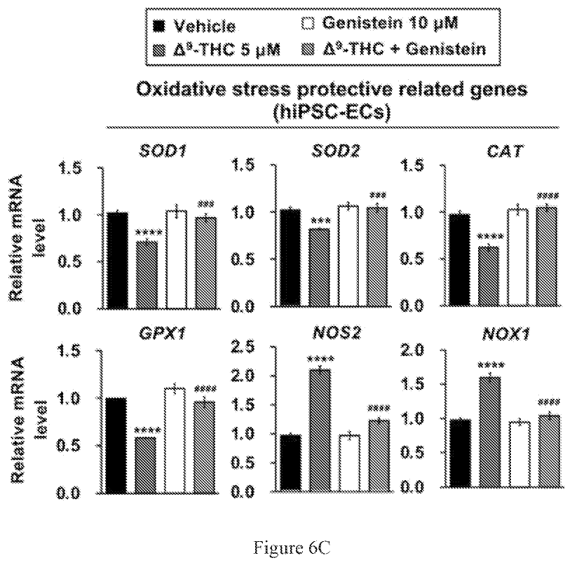

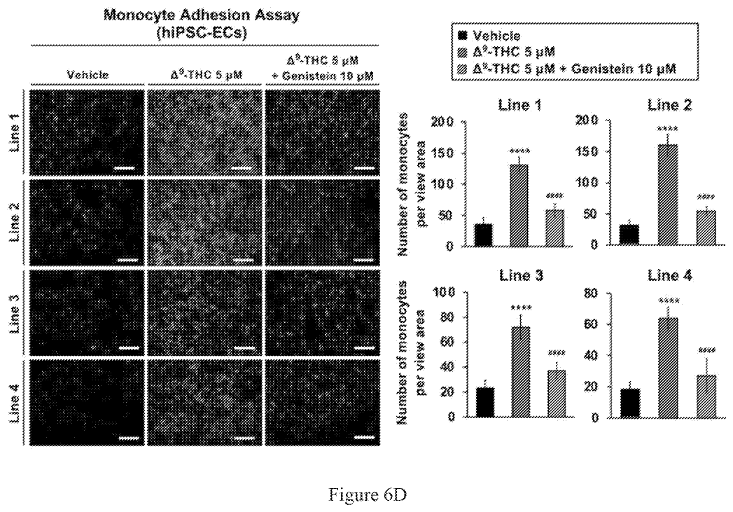

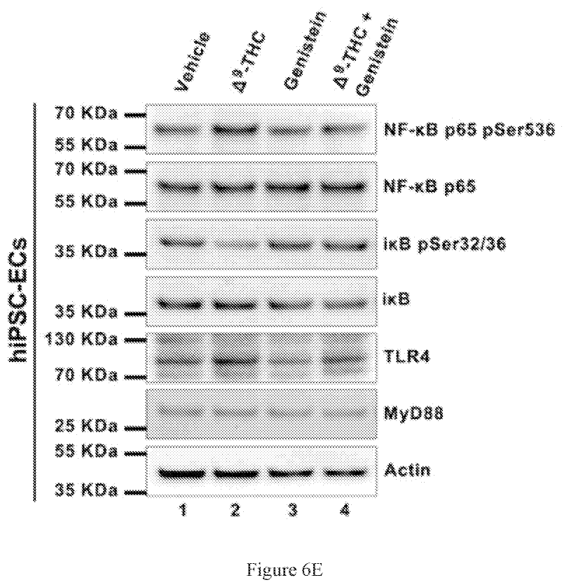

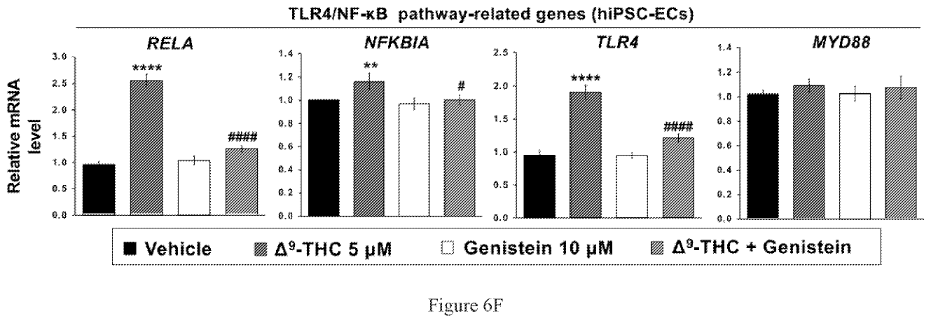

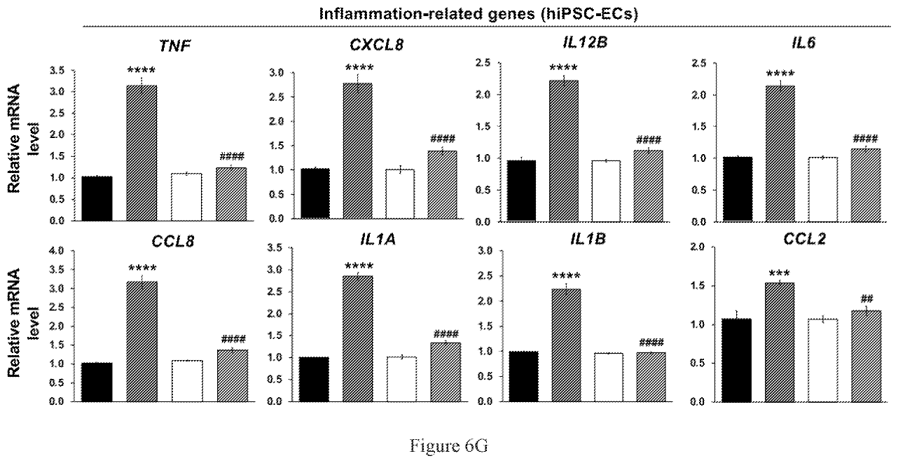

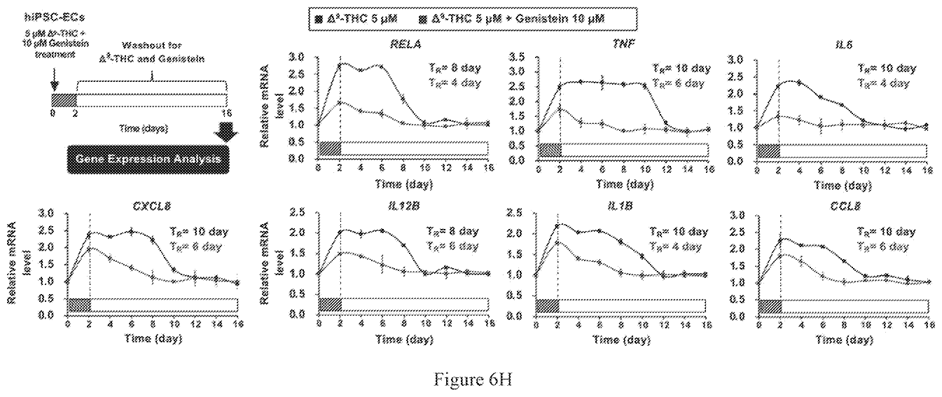

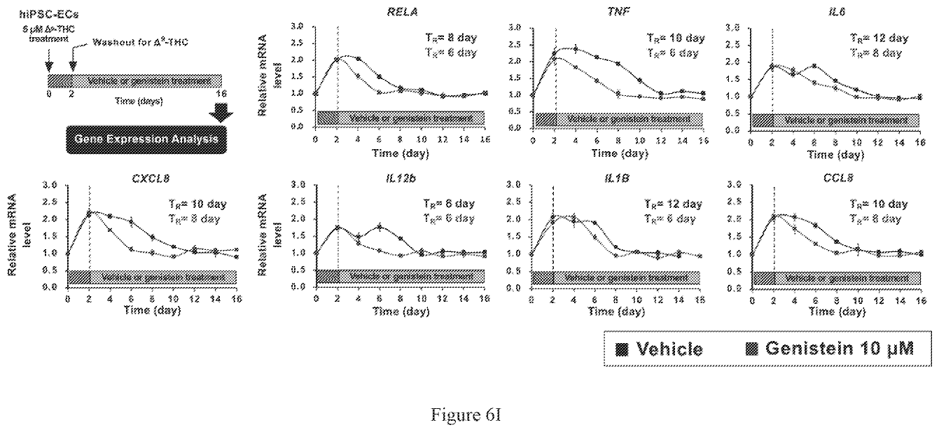

In this part, the inventor screened a series of antioxidant reagents and found that genistein was the best at preventing Δ⁹-THC-induced oxidative stress in hiPSC-ECs (FIGS. 6A and 6B). In hiPSC-ECs, genistein ameliorated the expression profile of antioxidant genes suppressed by Δ⁹-THC while also having a salutatory effect on NOS2 and NOX1 expression (FIG. 6C). The above experimental results confirmed that the putative CB1 antagonist genistein prevents vascular dysfunction caused by Δ⁹-THC.

With an attenuated inflammatory profile, the inventor postulated that hiPSC-ECs treated with genistein and Δ⁹-THC was more likely to remain quiescent without contributing to the pathogenesis of atherosclerosis. Genistein not only reversed Δ⁹-THC-induced monocyte adhesion to hiPSC-ECs but also attenuated NF-κB phosphorylation by using monocyte adhesion assays (FIGS. 6D and 6E).

Toll-like receptors (TLRs) are components of the innate immune system that recognize molecular patterns of microbial components, and TLR4/NF-κB signaling pathways can contribute to vascular inflammation in endothelial cells. Therefore, the inventor investigated the effects of Δ⁹-THC and the anti-inflammatory properties of genistein on the TLR4/NF-κB signaling pathway. As shown in the experimental results, Δ⁹-THC induced TLR4 expression in hiPSC-ECs, whereas genistein disrupted its expression (FIGS. 6E and 6F). Moreover, genistein reversed the Δ⁹-THC-induced mRNA expression of proinflammatory cytokines and chemokines (FIG. 6G). Cotreatment with genistein also shortened the recovery time of Δ⁹-THC-induced inflammation from 8-10 days to 4-6 days (FIG. 6H), and treatment with genistein after Δ⁹-THC exposure also reduced the recovery time to 6-8 days (FIG. 6I). These experimental results indicated that genistein attenuated Δ⁹-THC-induced oxidative stress and inflammation in hiPSC-ECs.

### EXAMPLE 8

### Effect of Genistein In Vivo

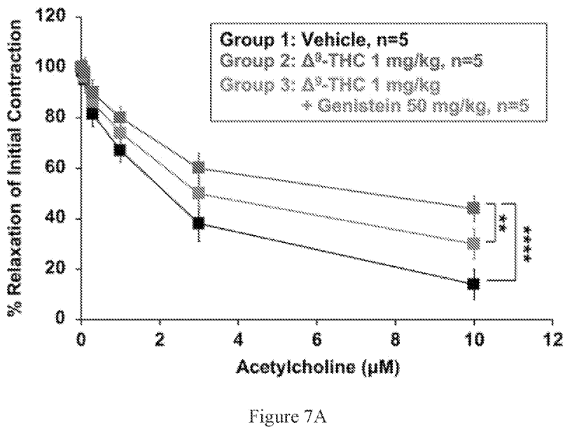

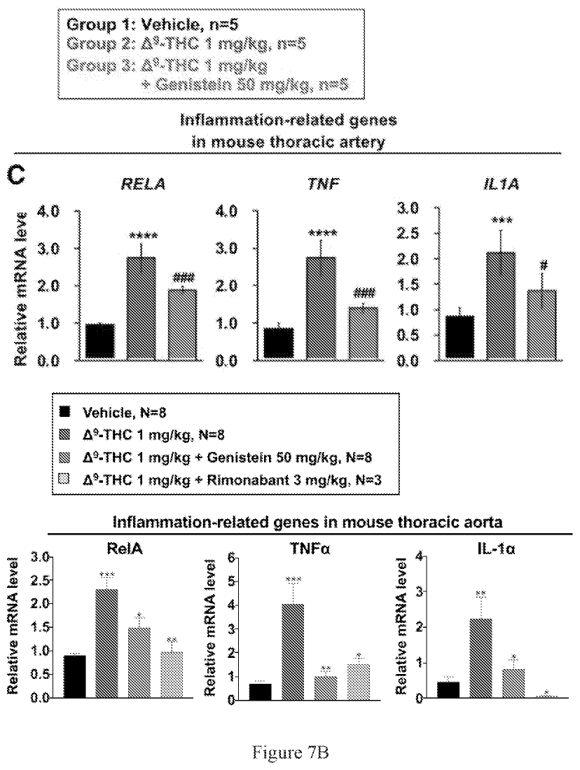

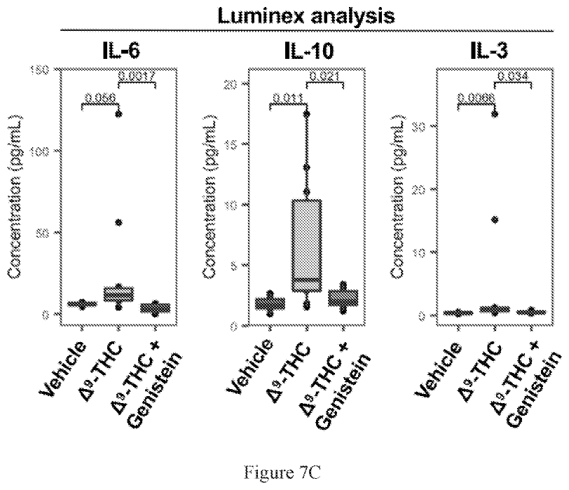

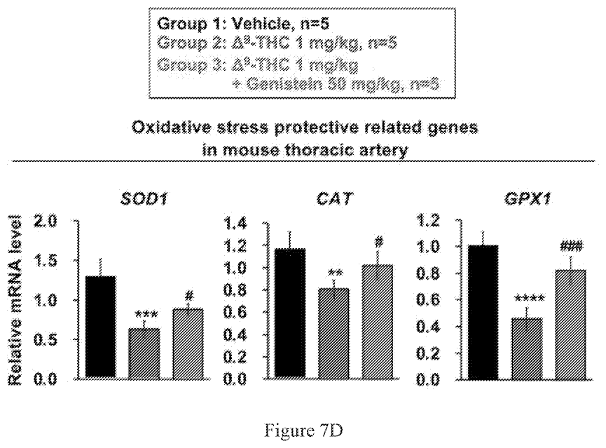

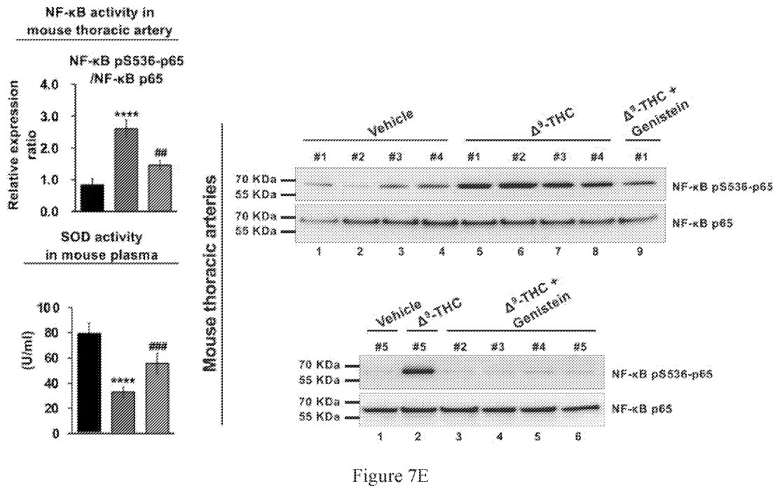

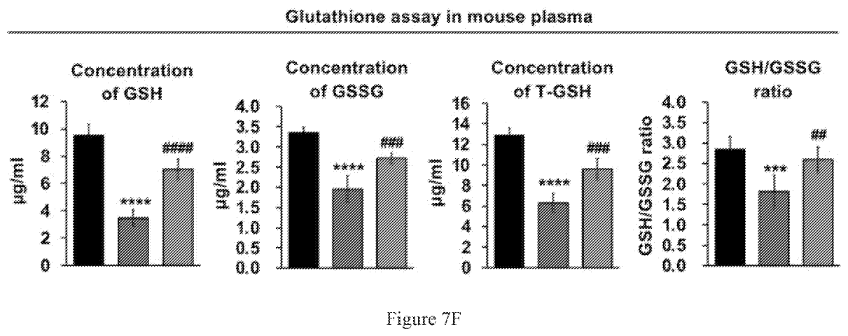

To evaluate the effects of genistein in vivo, male C57BL/6J mice were treated with vehicle (n=5), Δ⁹-THC (n=5, administered Δ⁹-THC 1 mg/kg), or Δ⁹-THC plus genistein (n=5, administered Δ⁹-THC 1 mg/kg plus genistein 50 mg/kg). The mouse model produced a plasma concentration of Δ⁹-THC of ˜100 ng/mL as revealed by LC-MS analysis (not shown in the figure), which is comparable to the concentration from smoking a single marijuana cigarette. Wire myograph revealed that Δ⁹-THC induced endothelial dysfunction in mice, whereas genistein mitigated the effect (FIG. 7A).

Genistein and the known CB1 antagonist rimonabant ameliorated mRNA expressions of inflammation-related genes induced by Δ⁹-THC in mouse thoracic artery tissues (FIG. 7B). Analysis of C57BL/6J mouse plasma revealed that IL-6, IL-3, and IL-10, which are associated with an increased risk of atherosclerosis, were elevated after treatment with Δ⁹-THC, and genistein cotreatment significantly reduced the expression of these inflammatory cytokines (FIG. 7C). Genistein also rescued the oxidative stress protective-related gene expression (FIG. 7D). In addition, NF-κB phosphorylation and SOD expression in mouse serum were also attenuated with genistein (FIG. 7E). Δ⁹-THC administration decreased circulating levels of the antioxidant glutathione, which was ameliorated with genistein cotreatment (FIG. 7F). Collectively, these results indicated that genistein could reverse Δ⁹-THC-induced effects in vivo.

### EXAMPLE 9

### Effects of Genistein and Δ⁹-THC on the Heart

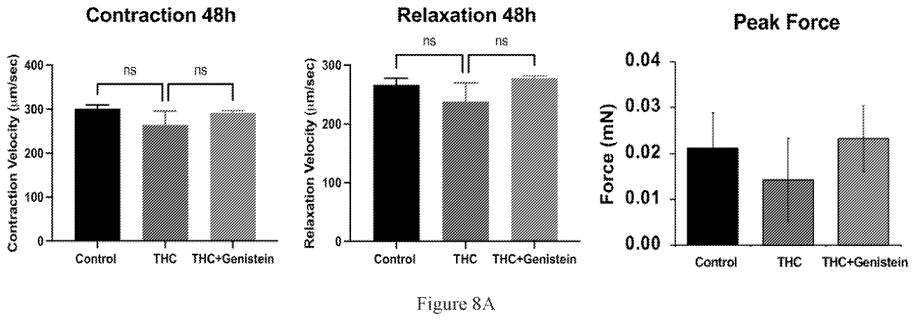

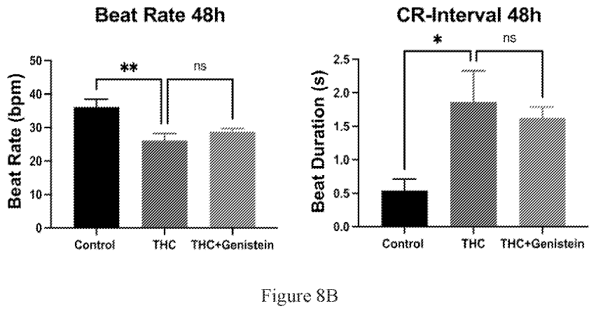

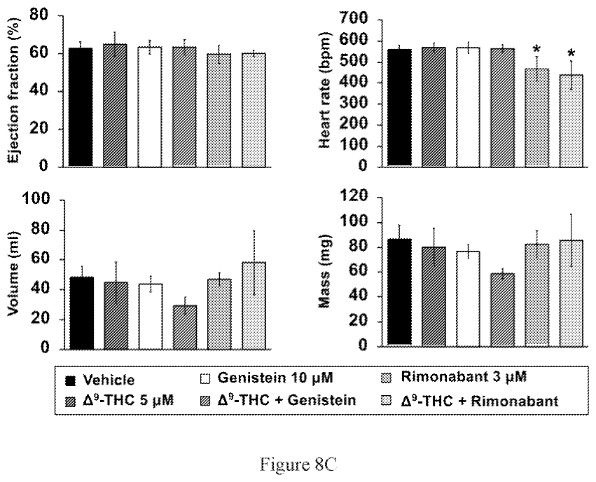

While genistein had a protective effect on the vasculature, it did not exhibit any toxicity to various organs (not shown in the figure). The inventor used engineered heart tissues (EHTs) composed of hiPSC-ECs and hiPSC-derived cardiomyocytes (hiPSC-CMs) to interrogate the effects of genistein and Δ⁹-THC on contractility in vitro. The results showed that Δ⁹-THC and genistein cotreatment had no significant effect on contraction, relaxation, and peak force (FIG. 8A). More specifically, Δ⁹-THC decreased beat rate and increased the contraction-relaxation interval, and genistein cotreatment did not attenuate these effects (FIG. 8B). Reflecting the EHT studies, echocardiographic analysis of C57BL/6J revealed that Δ⁹-THC, genistein, and cotreatment with genistein did not affect cardiac structure or function, with no significant difference in ejection fraction (EF), heart rate (HR), left ventricular end-diastolic volume (LVEDV), and left ventricular end-diastolic mass (LVEDM). The CB1 antagonist rimonabant, however, reduced HR when used alone or in cotreatment with Δ⁹-THC but did not affect other cardiac parameters (FIG. 8C).

### EXAMPLE 10

### Effects of Δ⁹-THC and Genistein on the Formation of Chronic Atherosclerosis

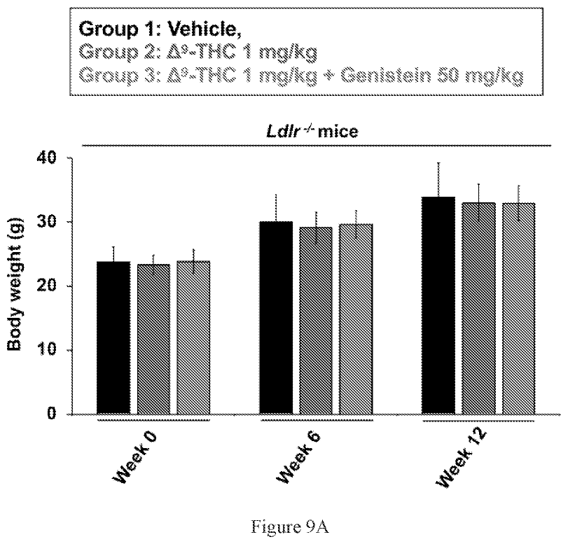

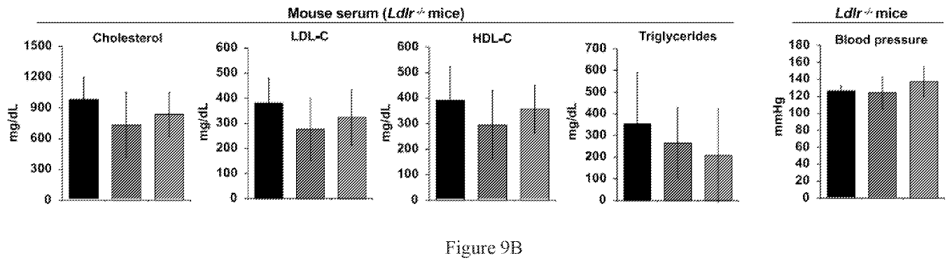

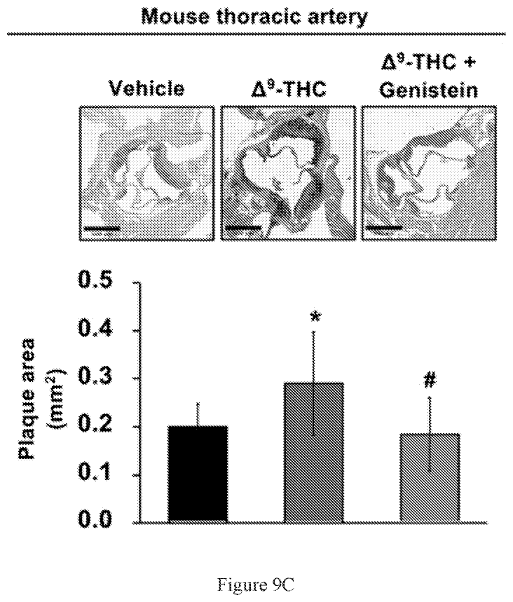

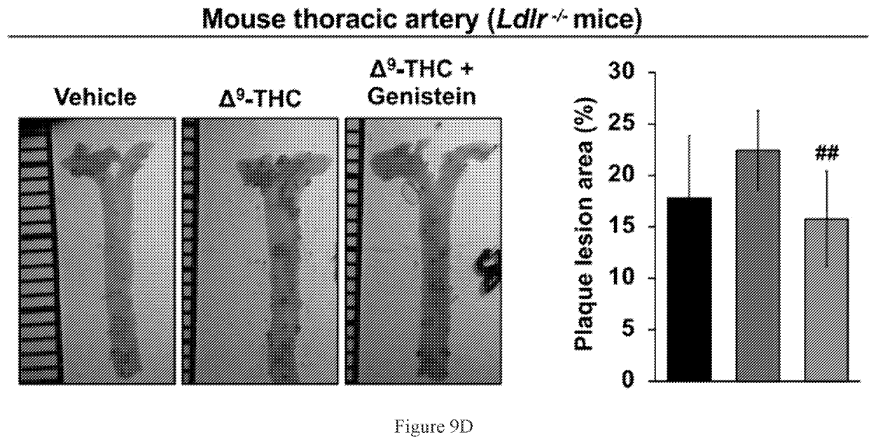

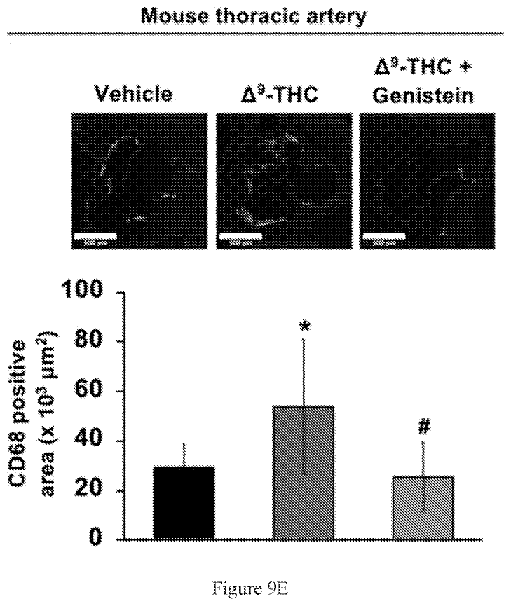

In this part, In order to investigate the effect of Δ⁹-THC and genistein in chronic atherosclerosis formation, LDL receptor knockout mice (B6.129S7-Ldlr^{tm1Her/J}) were fed a high-fat diet (HFD) for 12 weeks and treated with (1) vehicle control (n=10 to 12/group), (2) Δ⁹-THC (n=10 to 12/group, administered Δ⁹-THC 1 mg/kg), or (3) Δ⁹-THC and genistein (n=10 to 12/group, administered Δ⁹-THC 1 mg/kg plus genistein 50 mg/kg). All groups showed an increase in body weight during the HFD period (FIG. 9A). The successful administration of Δ⁹-THC was confirmed in serum by LC-MS at 12 weeks. Neither Δ⁹-THC nor cotreatment with Δ⁹-THC and genistein affected serum lipid profiles or blood pressure (FIG. 9B). After 12 weeks of HFD, the mice were euthanized, and the atherosclerotic lesion area was examined by cross-sections of the aortic root and an en face analysis of the thoracic aorta. In the cross-sectional analysis, mice treated with Δ⁹-THC showed significantly increased plaque size, and cotreatment with genistein ameliorated plaque size (FIG. 9C). The improvement of plaque formation by genistein was also observed in en face analysis of the thoracic aorta stained with oil red O (FIG. 9D). Macrophage recruitment in the aortic root was analyzed using aortic plaque stained with an anti-CD68 antibody. The CD68-positive area was significantly increased by Δ⁹-THC administration and rescued by cotreatment with genistein (FIG. 9E). These results suggest that Δ⁹-THC exacerbated atherosclerosis formation and macrophage recruitment in atherosclerotic plaques, which were ameliorated by genistein cotreatment.

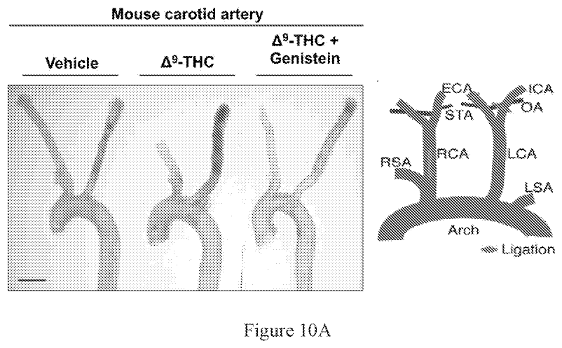

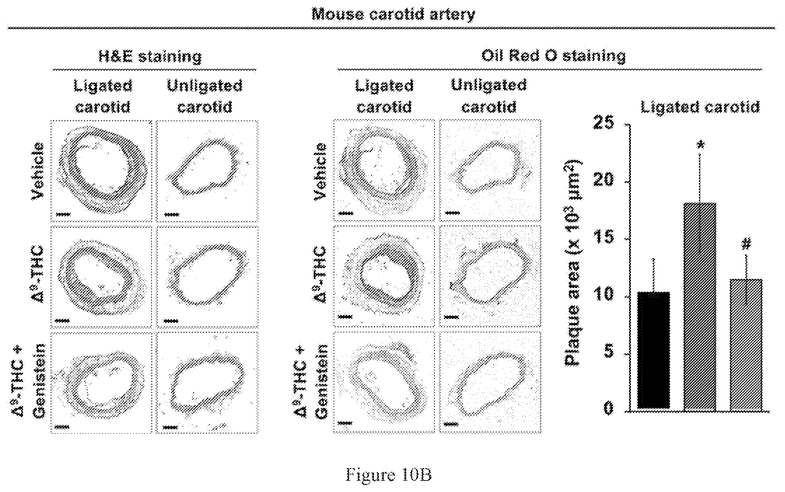

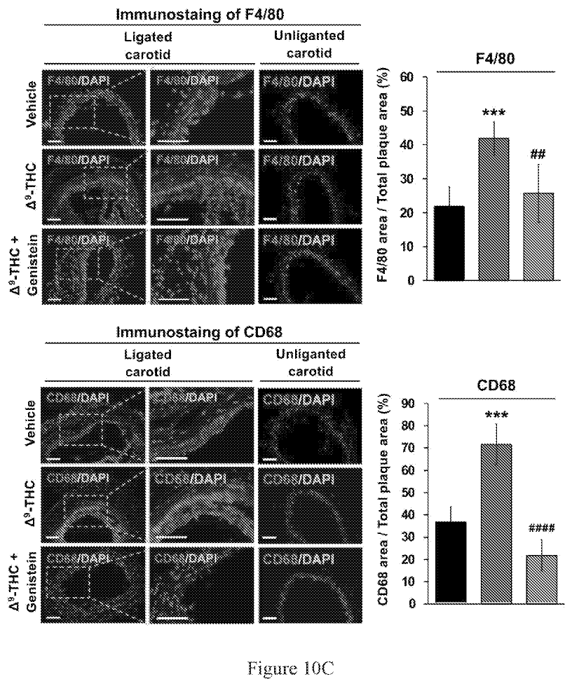

Further, an Apoe^{-/-} mouse model was also employed to investigate the effects of Δ⁹-THC and cotreatment with genistein on atherosclerosis. Partial carotid artery ligation (PCAL) was performed in Apoe^{-/-} mice at 10-16 weeks. After PCAL, the Apoe^{-/-} mice were divided into three groups: (1) vehicle control (n=5/group), (2) Δ⁹-THC (n=5/group, administered Δ⁹-THC 1 mg/kg), and (3) Δ⁹-THC plus genistein (n=5/group, administered Δ⁹-THC 1 mg/kg plus genistein 50 mg/kg). All mice were fed a HFD for 10 days. At the end of the treatment protocol, the Apoe^{-/-} mice were euthanized and subjected to histological analysis. The ligated carotid artery showed increased fat deposition with oil red O staining compared with the unligated contralateral vessel (FIG. 10A). H&E staining suggested neointimal thickening, fat deposition, and increased macrophage recruitment in ligated carotid atherosclerotic plaques. Oil red O staining found that the plaque area was increased by Δ⁹-THC treatment, whereas Δ⁹-THC and genistein cotreatment ameliorated the plaque size (FIG. 10B). Macrophage recruitment was tested using the macrophage-specific F4/80 antibody and CD68 antibody. As shown in FIG. 10C, in the ligated carotid artery, the F4/80-positive area was significantly increased by Δ⁹-THC administration and decreased by cotreatment with genistein. Immunohistochemical analysis with CD68-specific antibody revealed that the CD68-positive area was significantly increased by Δ⁹-THC administration and reduced by cotreatment with genistein. Consistent with the Ldlr^{-/-} model findings, the Apoe^{-/-} PCAL mouse model found increased plaque size and increased macrophage recruitment with Δ⁹-THC, and genistein cotreatment ameliorated these indices of atherosclerosis in ligated carotid arteries.

### EXAMPLE 11

### Binding of Genistein In Vivo and In Vitro

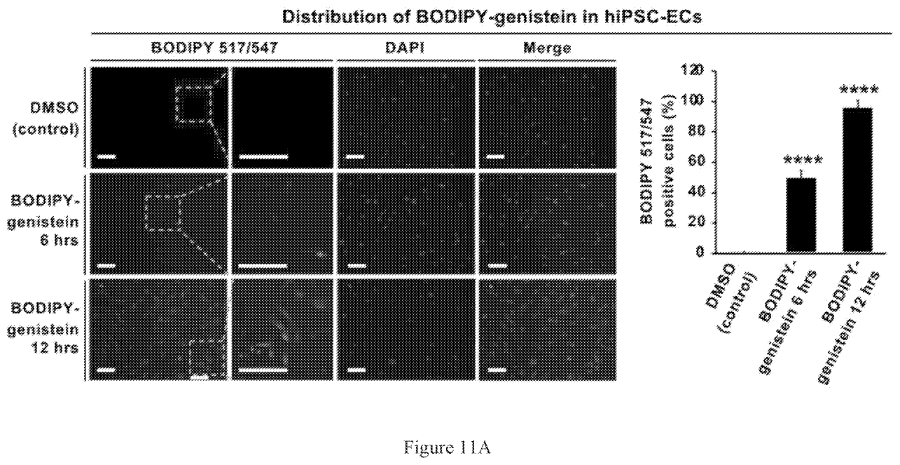

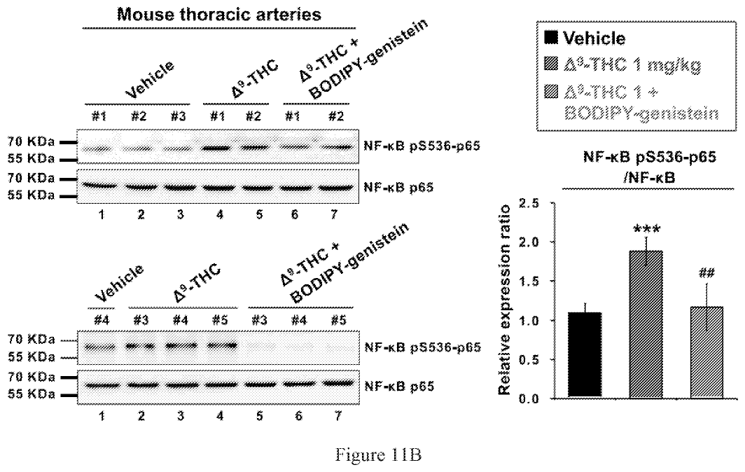

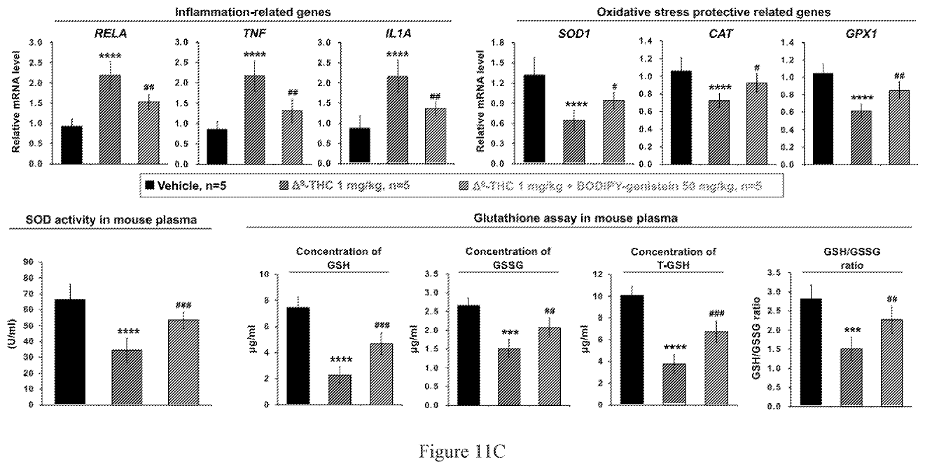

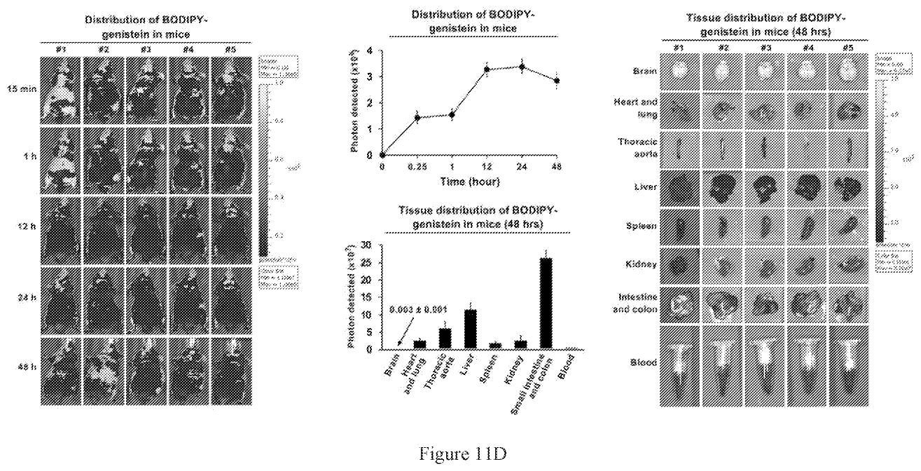

After finding that genistein attenuated the vascular dysfunction caused by Δ⁹-THC in both in vitro and in vivo studies, the inventor found direct evidence of genistein-binding CB1 using fluorescently labeled genistein (BODIPY517/547-genistein) (not shown in the figure). In CB1 radioligand binding assays, BODIPY517/547-genistein had an IC₅₀ of 375 nM on human CB1 receptors and labeled nearly all hiPSC-ECs cells after 12 hours incubation (FIG. 11A). BODIPY517/547-genistein cotreatment attenuates Δ⁹-THC-induced inflammation-related gene expression and NF-κB phosphorylation and ameliorates oxidative stress protective-related gene expression, SOD expression, and glutathione concentration in mouse serum (FIGS. 11B and 11C). In vivo binding was investigated using intravenously injected BODIPY517/547-genistein in C57BL/6J mice. After 48 hours, BODIPY517/547-genistein was detected in the abdominal viscera, thoracic aorta, heart, and lungs but was minimally detected in the brain (FIG. 11D), which shows genistein has poor blood-brain barrier (BBB) penetration.

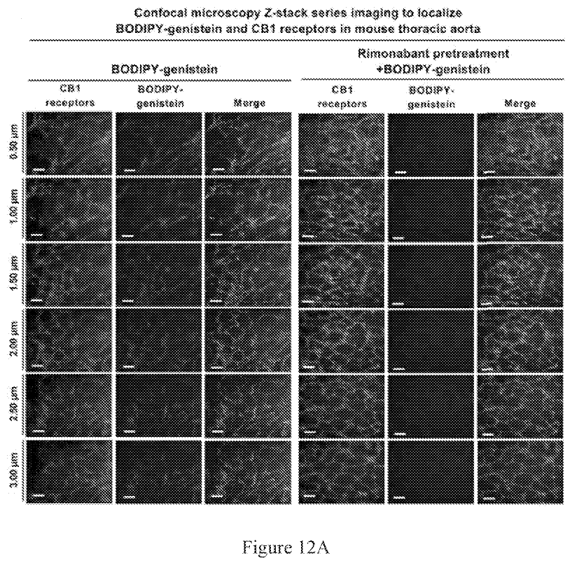

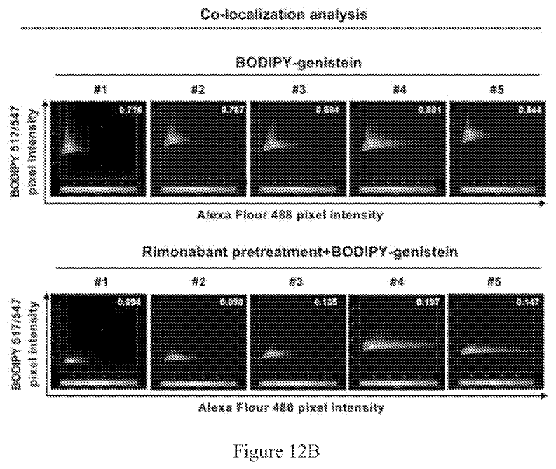

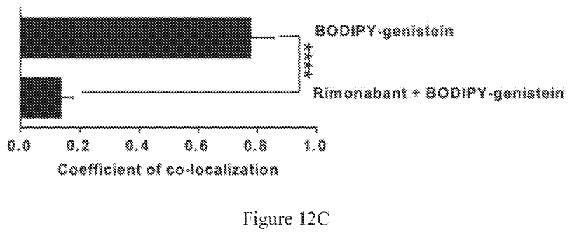

Further, the inventor investigated whether genistein is co-localized with the CB1 receptor in vivo using confocal microscopy. Thoracic aortas from mice injected with BODIPY517/547-genistein were permeabilized and stained with an anti-CB1 receptor antibody labeled with Alexa Fluor 488. The mice were imaged with confocal microscopy, and z stacked images revealed co-localization of Alexa Fluor-488-labeled CB1 receptor and BODIPY517/547-genistein in aortic vascular cells (FIGS. 12A and 12B). The specificity of genistein binding was interrogated by injecting the selective CB1 antagonist rimonabant before BODIPY517/547-genistein. Rimonabant has a stronger binding affinity for the CB1 receptor and reduced BODIPY517/547-genistein binding and fluorescence significantly (0.78±0.08 versus 0.13±0.04, p<0.00001) (FIG. 12C).

### EXAMPLE 12

### Effects of Genistein on the Neurobehavioral Effects of Δ⁹-THC

Despite BODIPY517/547 having excellent bioavailability, genistein has poor BBB penetration, which compelled the inventor to interrogate the neurological effects of Δ⁹-THC in the context of genistein co-administration. The neurobehavioral effects of Δ⁹-THC include decreased mobility, analgesia, hypothermia, and sedation. These are described as the Billy Martin tetrad.

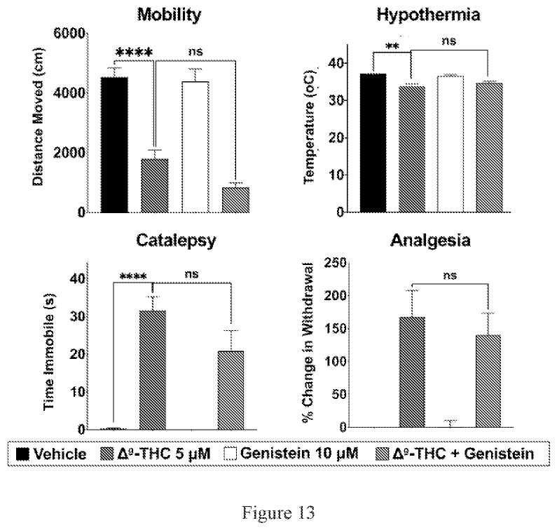

Therefore, the neurological effects of Δ⁹-THC and genistein co-administration were tested in C57BL/6J mice treated with vehicle, genistein, Δ⁹-THC, or in combination. The tetrad effects were assessed using an activity chamber for mobility, a hot plate for analgesia, a rectal probe for hypothermia, and the bar test for sedation. As shown in the FIG. 13, Δ⁹-THC caused decreased mobility, analgesia, hypothermia, and sedation in C57BL/6J mice. Genistein alone did not affect tetrad effects, and genistein could not attenuate the Δ⁹-THC neurobehavioral effects in cotreatment.

Based on the aforementioned experimental results, the present invention has first established the correlation between Δ⁹-THC and pathological conditions such as vascular inflammation, oxidative stress, endothelial cell dysfunction, and atherosclerosis. Furthermore, it has identified the CB1 receptor as a necessary factor for the effects of Δ⁹-THC. Additionally, it has been confirmed that genistein can bind to the CB1 receptor and inhibit its activity. Both in vitro and in vivo studies have confirmed genistein as a neutral antagonist of the CB1 receptor. Subsequent studies have demonstrated that genistein blocks the CB1 receptor and further alleviates the pathological effects caused by Δ⁹-THC in blood vessels, without penetrating the blood-brain barrier or affecting the neurobehavioral effects of Δ⁹-THC. Therefore, genistein can serve as a novel and advantageous CB1 receptor antagonist for the treatment or prevention of CB1 receptor-mediated diseases. Additionally, the present invention utilizes the phosphate ester derivatives of genistein to enhance bioavailability and water solubility, further enhancing the efficacy of genistein.

While a detailed description of the present invention has been given above, it should be understood that the foregoing embodiments are only some preferred ones of the invention and are not intended to be restrictive of the scope of the invention. Any equivalent change or modification that is based on the appended claims shall fall within the scope of the invention.

What is claimed is:

1. A use of a genistein and its phosphate ester derivative for the preparation of a medicament for treatment or prevention of diseases mediated by cannabinoid receptor type 1 (CB1 receptor) in an individual.

2. The use of claim 1, wherein the treatment or prevention of CB1 receptor-mediated diseases in the individual involves alleviating oxidative stress and endothelial dysfunction induced by Δ⁹-tetrahydrocannabinol, dronabinol, or nabilone.

3. The use of claim 2, wherein the oxidative stress involves a decrease in the expression levels of antioxidant genes and an increase in the expression levels of reactive oxygen species (ROS)-related genes: the antioxidant genes include superoxide dismutase 1 (SOD1) gene, superoxide dismutase 2 (SOD2) gene, catalase (CAT) gene, or glutathione peroxidase 1 (GPX1) gene, and the ROS-related genes involve the expression of NADPH oxidase 1 (NOX1) or inducible nitric oxide synthase (NOS2).

4. The use of claim 1, wherein the medicament alleviates inflammation induced by Δ⁹-tetrahydrocannabinol, dronabinol, or nabilone.

5. The use of claim 4, wherein the medicament reduces mRNA expression levels of pro-inflammatory cytokines and chemokines induced by Δ⁹-tetrahydrocannabinol, dronabinol, or nabilone.

6. The use of claim 1, wherein the CB1 receptor-mediated diseases are diseases or conditions associated with the coupling of CB1 receptor with Δ⁹-tetrahydrocannabinol, dronabinol, or nabilone.

7. The use of claim 1, wherein the CB1 receptor-mediated diseases include cardiovascular diseases, mental disorders, anxiety disorders, depression, epilepsy, neurodegenerative diseases, cognitive impairment, brain trauma, glaucoma, Parkinson's disease, Alzheimer's disease, Huntington's chorea, tremors, senile dementia, Tourette syndrome, cancer, hypotonia, septic shock, hypotension, multiple sclerosis, vomiting, asthma, eating disorders, obesity, diabetes, diseases associated with demyelination, neuroinflammation, viral encephalitis, liver cirrhosis, or gastrointestinal diseases.

8. The use of claim 7, wherein the cardiovascular diseases include atherosclerosis, coronary heart disease, peripheral vascular disease, myocardial infarction, and carotid artery stenosis.

9. A pharmaceutical composition for treatment or prevention of CB1 receptor-mediated diseases, comprising an effective amount of genistein and its phosphate ester derivative.

10. The pharmaceutical composition of claim 9, further comprising at least one other therapeutic agent.

11. The pharmaceutical composition of claim 10, wherein the at least one other therapeutic agent is anti-obesity agents, appetite suppressants, anti-diabetic agents, anti-hyperlipidemic agents, lipid-lowering agents, blood cholesterol-lowering agents, fat-modulating agents, cholesterol-lowering agents, fat-reducing agents, high-density lipoprotein-raising agents, anti-hypertensive agents, preparations for treating sleep disorders, preparations for treating substance abuse or addiction disorders, anti-anxiety agents, antidepressants, antipsychotic agents, cognitive enhancers, preparations for treating cognitive disorders, preparations for treating Alzheimer's disease, preparations for treating Parkinson's disease, anti-inflammatory agents, preparations for treating neurodegenerative disorders, preparations for treating arteriosclerosis, preparations for treating respiratory conditions, preparations for treating intestinal diseases, preparations for treating liver cirrhosis, or antineoplastic agents.

12. The pharmaceutical composition of claim 10, wherein said pharmaceutical composition is used for the treatment of chronic dependence, alcohol dependence, or drug abuse.

13. The pharmaceutical composition of claim 12, wherein said pharmaceutical composition is used for the treatment of cannabis abuse.

14. The pharmaceutical composition of claim 10, wherein said pharmaceutical composition enhances the analgesic activity of analgesic drugs or anesthetic drugs.

15. The pharmaceutical composition of claim 9, wherein said phosphate ester derivative of genistein is genistein 7-O-phosphate (G7P) or genistein 4′-O-phosphate (G4′P).

16. The pharmaceutical composition of claim 9, wherein said phosphate ester derivative of genistein is obtained by microbial fermentation of genistein or by contacting or culturing genistein with a polyphenolic flavonoid phosphate synthase.

17. The pharmaceutical composition of claim 16, wherein said microorganism comprises a nucleic acid sequence encoding a polypeptide having a homologous protein sequence with a similarity of at least 70% to the polyphenolic flavonoid phosphate synthase (SEQ ID NO: 1), said polypeptide comprising a conserved region, said conserved region being based on SEQ ID NO: 1 and sequentially comprising:
an ATP binding region, including active catalytic sites of lysine 27 (Lys27), arginine 102 (Arg102), and glutamic acid 282 (Glu282);
a substrate binding region, including a conserved motif DDHHFYIDAMLDAKAR (SEQ ID NO: 2), and active catalytic sites of aspartic acid 627 (Asp627), histidine 629 (His629), and histidine 630 (His630); and
a phosphorylated histidine catalytic region, including an active catalytic site of histidine 795 (His795).
